(19) United States
(12) Patent Application Publication
(10) Pub. No.: US 2021/0041447 A1
(43) Pub. Date: Feb. 11, 2021

(54) AZACARBOLINE COMPOUNDS FOR THE DETECTION OF TAU AGGREGATES

(71) Applicants: AC IMMUNE SA, Lausanne (CH); LIFE MOLECULAR IMAGING SA, Matran (CH)

(72) Inventors: Mathias BERNDT, Berlin (DE); Andre MÜLLER, Berlin (DE); Felix ODEN, Berlin (DE); Hanno SCHIEFERSTEIN, Wiesbaden (DE); Heribert SCHMITT-WILLICH, Berlin (DE); Heiko KROTH, Ecublens (CH); Jérôme MOLETTE, Prevessin Moens (FR); Emanuele GABELLIERI, Lausanne (CH); Cédric BOUDOU, Reignier (FR)

(21) Appl. No.: 16/964,964

(22) PCT Filed: Jan. 22, 2019
(86) PCT No.: PCT/EP2019/051496

§ 371 Date: Jul. 24, 2020

(30) Foreign Application Priority Data
Jan. 24, 2018 (EP) 18153323.3

(51) Int. Cl. G01N 33/60; A61K 51/04; C07D 471/14; C07B 59/00; G01N 33/68

(52) U.S. Cl. CPC G01N 33/60; A61K 51/0455; C07D 471/14; G01N 2800/2821; G01N 33/6896; C07B 2200/05; C07B 59/002

(57) ABSTRACT

The present invention relates to novel compounds of the formula (II)

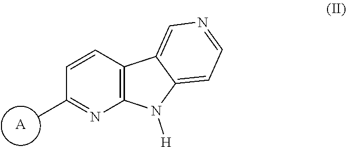

that can be employed in the selective Tau detection of disorders and abnormalities associated with Tau aggregates such as Alzheimer's disease and other tauopathies using Positron Emission Tomography (PET) Imaging. The present invention also discloses intermediates which are useful in the preparation of these compounds.

### FIELD OF INVENTION

The present invention relates to novel compounds of the formula (II) that can be employed in the selective detection of disorders and abnormalities associated with Tau aggregates such as Alzheimer's disease (AD) and other tauopathies, for example, using Positron Emission Tomography (PET) imaging. The present invention also refers to intermediates of the formula (III) which can be used in the production of such imaging compounds. Diagnostic compositions as well as methods of imaging or diagnosing using the above compounds and kits which are useful for preparing a radiopharmaceutical preparation are also subject of the present invention.

### BACKGROUND

Alzheimer's disease is a neurological disorder primarily thought to be caused by amyloid plaques, an extracellular accumulation of abnormal deposit of amyloid-beta (Aβ) aggregates in the brain. The other major neuropathological hallmarks in AD are the intracellular neurofibrillary tangles (NFT) that originate by the aggregation of the hyperphosphorylated Tau (Tubulin associated unit) protein, phosphorylated Tau or pathological Tau and its conformers. AD shares this pathology with many neurodegenerative tauopathies, in particularly with specified types of frontotemporal dementia (FTD). In AD brain, Tau pathology (tauopathy) develops later than amyloid pathology, but it is still discussed controversially if Aβ protein is the causative agent in AD which constitutes the essence of the so-called amyloid cascade hypothesis (Hardy et al., Science 1992, 256, 184-185, and most recently, Musiek et al., Nature Neurosciences 2015, 18(6), 800-806, “Three dimensions of the amyloid hypothesis: time, space and ‘wingmen’”).

Presently, the only definite way to diagnose AD is to identify plaques and tangles in brain tissue by histological analysis of biopsy or autopsy materials after the death of the individual. Beside AD, Tau plays an important role in other (non-AD) neurodegenerative diseases. Such non-AD tauopathies include, for example, supranuclear palsy (PSP), Pick's disease (PiD) and corticobasal degeneration (CBD).

Therefore, there is a great deal of interest in detection of Tau pathology in vivo. Tau PET imaging promises novel insights into deposition of Tau aggregates in the human brain and might allow to non-invasively examine the degree of Tau pathology, quantify changes in Tau deposition over time, assess its correlation with cognition and analyze the efficacy of an anti-Tau therapy. For recent reviews see Shah et al., J Nucl Med. 2014, 55(6), 871-874: “Molecular Imaging Insights into Neurodegeneration: Focus on Tau PET Radiotracers”, Jovalekic et al., EJNMMI Radiopharmacy and Chemistry 2016, 1:11, “New protein deposition tracers in the pipeline”, and Ariza et al., J Med Chem 2015, 58(11), 4365-82: “Tau PET Imaging: Past, Present and Future”. In addition, several patent applications have recently been published, e.g: WO 2013/176698, WO 2009/102498, WO 2011/119565, U.S. Pat. No. 8,932,557 B2 and U.S. Pat. No. 8,691,187,B2 (Siemens Medical Solutions, Lilly), WO 2012/067863 and WO 2012/068072 (both GE Healthcare) WO 2014/026881, WO 2014/177458, WO 2014/187762, WO 2015/044095, WO 2015/052105, WO 2015/173225 (Hoffmann-La Roche AG), WO 2015/188368 (Merck Sharp & Dohme) which claim novel compounds for Tau imaging.

In order to achieve high target selectivity, molecular probes have been used which recognize and bind to the pathological target. Selectivity for binding to pathological Tau protein over other protein depositions in the brain is therefore a basic requirement of a Tau imaging probe. In order to reduce background signal interference resulting from non-specific off-target binding (e.g. binding to Aβ or monoamine oxidases), imaging compounds should bind with high affinity to pathological Tau. Since amyloid or amyloid-like deposits formed from proteins of diverse primary amino acid sequences share a common p-sheet quaternary conformation, molecular probes are required that can differentiate such structures in order to avoid detection of other pathologies (false-positives) and therefore misdiagnosis.

Off-target binding to monoamine oxidase A or B have been reported to be a significant limitation for Tau tracers, especially T-807 and THK-5351 (Vermeiren, C, et al. Alzheimers & Dementia. 2015; 11 (7) Supplement p1-2: “T807, a reported selective tau tracer, binds with nanomolar affinity to monoamine oxidase A”; Ng, K P, et al. Alzheimer's Research and Therapy 2017, 9:25: “Monoamine oxidase B inhibitor, selegiline, reduces ¹⁸F-THK5351 uptake in the human brain”). Off-target binding to monoamine oxidases A or B confound the interpretation of PET images with T807 and THK5351 with respect to tau. Presence of monoamine oxidases within several brain regions limits the interpretation of PET imaging results with these tracers.

Beside high selectivity, also binding to different Tau isoforms is an important aspect for a tau tracer. Up till now, most tracers show binding to tau in AD. However, tau in AD is a mixture of two isoforms, so called 3R-tau and 4R-tau. Other non-AD tauopathies are characterized by the presence of only one isoform. In Pick's disease (PiD), the 3R tau isoform is present whereas in progressive supranuclear palsy (PSP) and in corticobasal degeneration (CBD), the 4R-tau isoform is the existing pathology.

In addition, molecular probes must also be designed such that upon administration they can distribute within the body and reach their target. For imaging of Tau aggregates associated with neurological disorders such as e.g. Alzheimer's disease, imaging compounds are required that can penetrate the blood brain barrier and pass into the relevant regions of the brain. For targeting intracellular Tau aggregates, cell permeability is an additional requirement of imaging compounds. A further prerequisite in order to get a sufficient signal-to-noise ratio is a fast compound wash-out from non-target regions in the brain (or other targeting organ). Also, compounds should show no defluorination, as bone uptake in the skull (as result from presence of free fluoride) will cause significant spill-over into the brain which limits the usability (Chien D T, et al. J Alzheimers Dis. 2014; 38:171-84).

The specifically disclosed and most advanced derivative of WO 2013/176698 is 2,5-disubstituted pyridine compound ¹⁸F-1 (also see U.S. Pat. No. 8,932,557 B2).

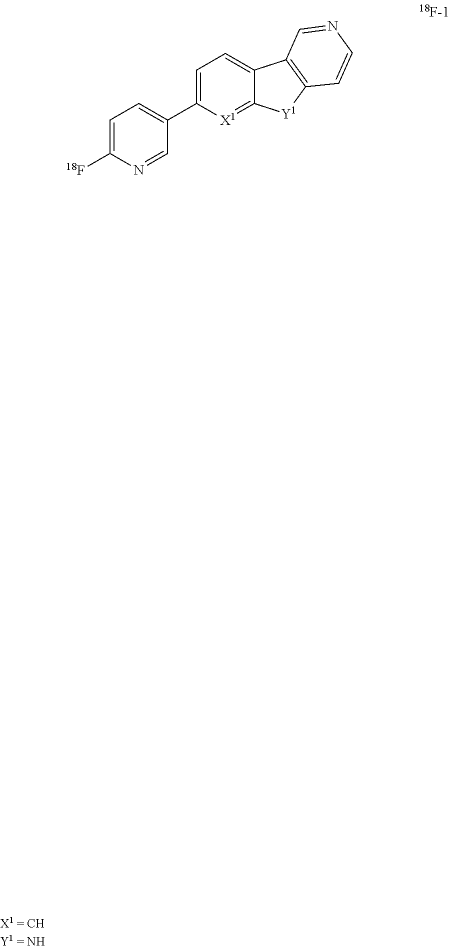

Compound ¹⁸F-1 was investigated in various clinical studies. Although ¹⁸F-1 seems to be able to detect Tau in patients with AD or amyloid-beta positive mild cognitive impairment (MCI), various limitations have been reported.

Vermeiren and coworkers found that compound ¹⁸F-1 bound to Monoamine oxidase A (MAO A) with a K_{D} of 1.5 nM. Their data unanimously demonstrate that compound ¹⁸F-1 binds to Tau aggregates and MAO-A with similar high affinity. The findings raise caution to the interpretation of compound ¹⁸F-1 clinical data, as MAO-A is widely expressed in most human brain regions (Vermeiren et al., Alzheimers & Dementia. 2015; 11 (7) Supplement p1-2:T807-a reported selective Tau tracer, binds with nanomolar affinity to Monoamine oxidase A).

Compound ¹⁸F-1 was reported to have a fairly strong signal in parts of the brain's basal ganglia, e.g., the striatum and substantia nigra, regardless of the patient's diagnosis. The signal of ¹⁸F-1 in the cortex did not reach a “steady state” (a window of time during which the ratio of binding in a target region to binding in the reference tissue (i.e. cerebellum) was stable). In addition, the kinetics of ¹⁸F-1 in various brain regions was different and never stabilized in a 150-minute scanning period (S. Baker, Human Amyloid Imaging Meeting, 2015).

Binding of compound ¹⁸F-1 to AD brain sections was demonstrated by autoradiography. However, compound ¹⁸F-1 showed limitations in binding to brain sections with pathologies of non-AD tauopathies: a) Lowe V J, et al. An autoradiographic evaluation of AV-1451 Tau PET in dementia. Acta Neuropathologica Communications. 2016; 4:58; b) Marquie M, et al. Validating novel Tau Positron Emission Tomography Tracer [F-18]-AV-1451 (T807) on postmortem Brain Tissue. Annals of Neurology. 2015; 78:787; c) Gomez F, et al. Quantitative assessment of [¹⁸F]AV-1451 distribution in AD, PSP and PiD Post-Mortem Brain Tissue Sections relative to that of the anti-Tau antibody AT8. Journal of Nuclear Medicine. 2016; 57, S2: 348, d) Sander K, et al. Characterization of tau positron emission tomography tracer AV1451 binding to postmortem tissue in Alzheimer's disease, primary tauopathies, and other dementias. Alzheimers Dementia 2016, 12(11): 116-1124 e) Smith R, et al. Increased basal ganglia binding of 18F-AV-1451 in patients with progressive supranuclear palsy. Movement disorders 2016.

Also clinically, ¹⁸F-1 seems to be of limited value for the detection of tau in PSP subjects: a) Smith R et al., Tau neuropathology correlates with FDG-PET, but nor with AV-1451-PET, in progressive supranuclear palsy. Acta Neuropathologica 2017, 133:149-151; b) Smith R, et al. Increased basal ganglia binding of 18F-AV-1451 in patients with progressive supranuclear palsy. Movement disorders 2017, 32(1), 108-114.

The final conclusions from these studies indicate that T807/AV1451 might not reliable to distinguish individual patients with PSP from controls. This is mainly attributed to an increased unspecific binding in midbrain structures like basal ganglia. Uptake seen in cerebral cortex and white matter did not reflected tau pathology in PSP.

Compound ¹⁸F-2 is disclosed in WO 2015/052105.

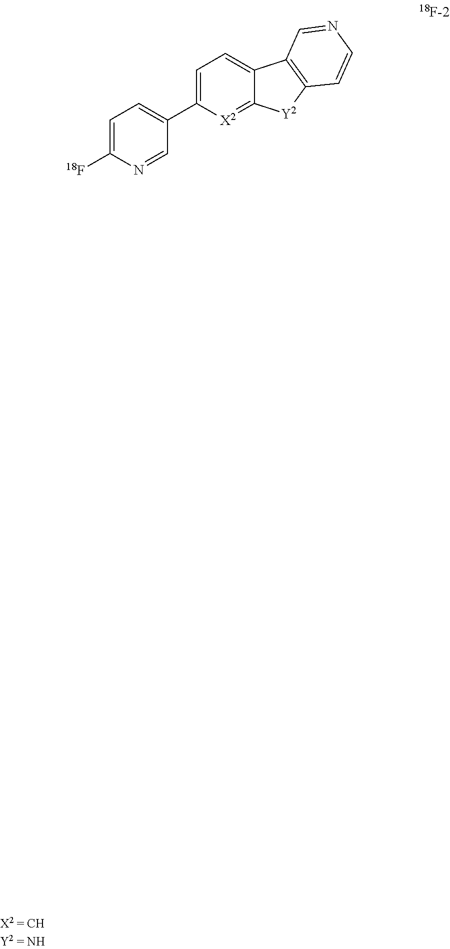

WO 2015/052105 only discloses one ¹⁸F-labeled compound and a corresponding compound which is tritium labeled. The compound comprises a 2,5-disubstituted pyridine moiety (compound ¹⁸F-2). WO 2015/052105 does not provide any data on binding to Tau-isoforms in non-AD tauopathies, binding to MAO A (or otherwise on selectivity to Tau), brain uptake, brain washout or retention in healthy brain, or any data on in vivo de-fluorination.

¹⁸F-2 was found to not bind to brain tissue from patients with non-AD tauopathies such as Pick's disease (PiD) and progressive supranuclear palsy (PSP) (Honer M et al., In vitro binding of ³H-RO6958948, ³H-AV-1451, ³H-THK5351 and ³H-T808 to tau aggregates in non-AD tauopathies. Human Amyloid Imaging 2017, abstract 99).

In view of the above mentioned prior art, it was an object of the present invention to provide a compound which has a high affinity and selectivity for Tau and is thus suitable as a PET imaging agent. Preferably, the compounds of the present invention demonstrate high affinity to Tau aggregates, high selectivity towards pathological Tau compared to other targets in the brain and favorable pharmacokinetic properties without defluorination. The desired Tau PET imaging agent should bind to both, 3R and 4R Tau to address AD and non-AD tauopathies including PiD, CBD and PSP.

### SUMMARY OF THE INVENTION

Therefore, the present invention relates to the following items:
1. A compound of the formula (I)

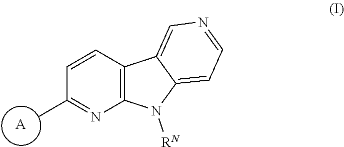

as well as pharmaceutically acceptable salts, hydrates, solvates, prodrugs and polymorphs thereof;
wherein

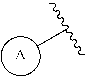

is selected from

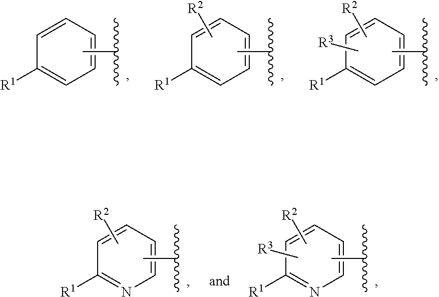

R¹ is selected from the group consisting of ¹⁸F, ¹⁹F and LG,
R² and R³ are independently selected from the group consisting of halogen, alkyl, alkoxy, —NR⁷R⁸, —N(R⁷)alkyl, —N(alkyl)₂, and cyano, wherein the alkyl group(s) in alkyl, alkoxy, —N(R⁷)alkyl and —N(alkyl)₂ are independently optionally substituted with one or more halogen(s),
R⁷ and R⁸ are each independently selected from the group consisting of hydrogen and PG1,
R^{N} is selected from the group consisting of hydrogen and PG2,
LG is a leaving group, and
PG1 and PG2 are independently selected from amine protecting groups.
2. The compound according to item 1, wherein

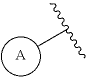

is selected from

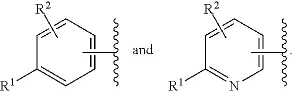

3. The compound according to item 1 or 2, wherein
   R² and R³ are independently selected from the group consisting of halogen, —NR⁷R⁸, —N(R⁷)alkyl, —N(alkyl)₂, and cyano, wherein the alkyl group(s) in —N(R⁷)alkyl and —N(alkyl)₂ are independently optionally substituted with one or more halogen(s), preferably R² and R³ are independently selected from the group consisting of halogen and cyano.
4. The compound according to item 1, wherein the compound has the formula (II)

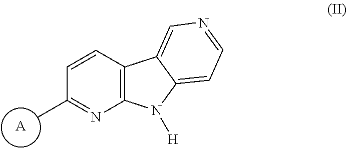

as well as pharmaceutically acceptable salts, hydrates, solvates, prodrugs and polymorphs thereof;
wherein

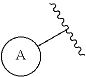

is selected from

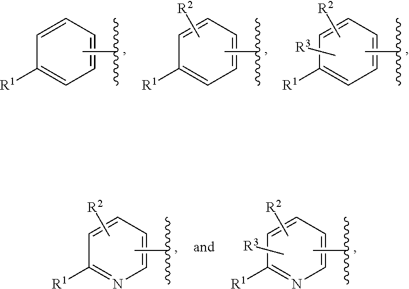

R¹ is selected from the group consisting of ¹⁸F and ¹⁹F, and
R² and R³ are independently selected from the group consisting of halogen, alkyl, alkoxy, —NH₂, —N(H)alkyl, —N(alkyl)₂, and cyano, preferably R² and R³ are independently selected from the group consisting of halogen, —NH₂, —N(H)alkyl, —N(alkyl)₂, and cyano, more preferably R² and R³ are independently selected from the group consisting of halogen and cyano,
wherein the alkyl group(s) in alkyl, alkoxy, —N(H)alkyl and —N(alkyl)₂ are independently optionally substituted with one or more halogen(s).
5. The compound according to item 4, wherein

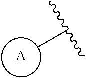

is selected from

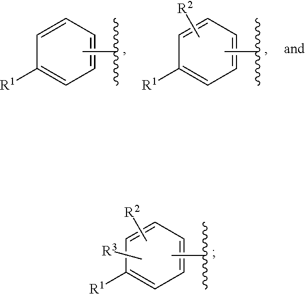

and
R¹, R² and R³ are as defined in item 4.
6. The compound according to item 5, wherein

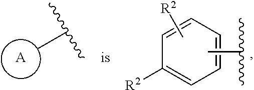

and
R¹ and R² are as defined in item 4.
7. The compound according to item 6, wherein the compound is selected from

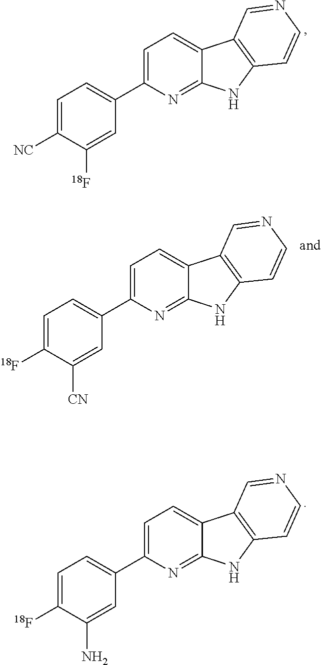 [SMILES: N#Cc1ccc(-c2ccc3c(n2)[nH]c2ccncc23)cc1[18F] ; N#Cc1cc(-c2ccc3c(n2)[nH]c2ccncc23)ccc1[18F] ; Nc1cc(-c2ccc3c(n2)[nH]c2ccncc23)ccc1[18F]]

8. The compound according to item 6, wherein
   R¹ is ¹⁸F, and
   R² is selected from the group consisting of halogen, alkyl, alkoxy, —NH₂, and cyano, wherein the alkyl group(s) in alkyl and alkoxy are independently optionally substituted with one or more halogen(s), preferably R² is selected from the group consisting of halogen, —NH₂, and cyano, more preferably R² is selected from the group consisting of halogen and cyano.
9. The compound according to item 4, wherein

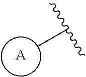

is selected from

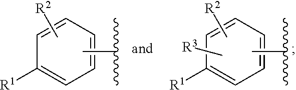

and
R¹, R² and R³ are as defined in item 4.
10. The compound according to item 9, wherein

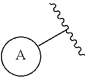

is selected from

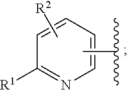

and
R¹ and R² are as defined in item 4.
11. The compound according to item 4, wherein the compound has the formula (IIa)

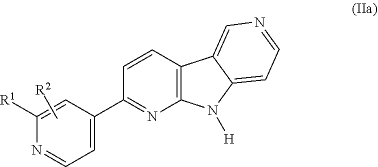

wherein R¹ and R² are as defined in item 4.
12. The compound according to item 11, wherein the compound is selected from

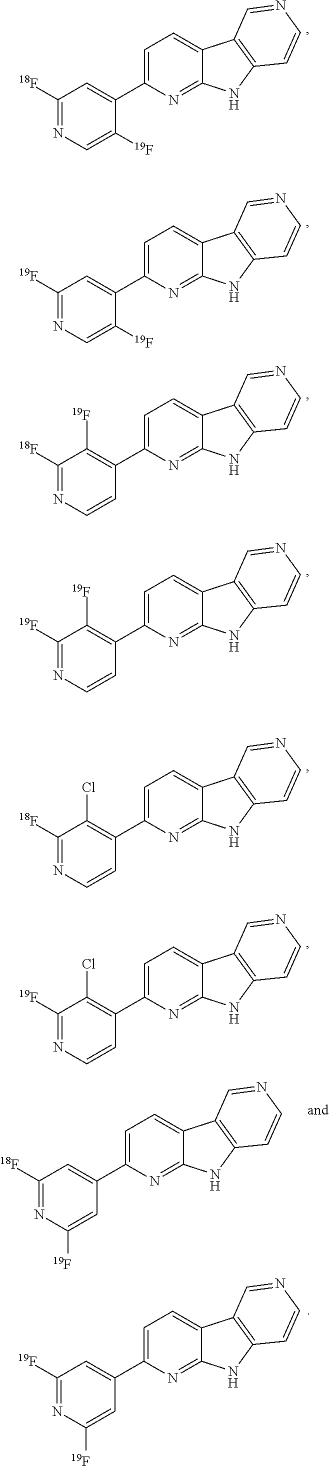

13. The compound according to item 11, wherein
   R¹ is ¹⁸F, and
   R² is as defined in item 4.
14. The compound according to item 4, wherein the compound has the formula (IIb)

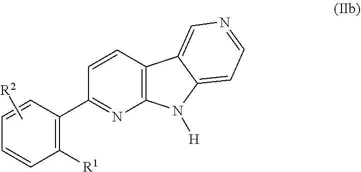

wherein
R¹ and R² are as defined in item 4.
15. The compound according to item 14, wherein the compound is selected from

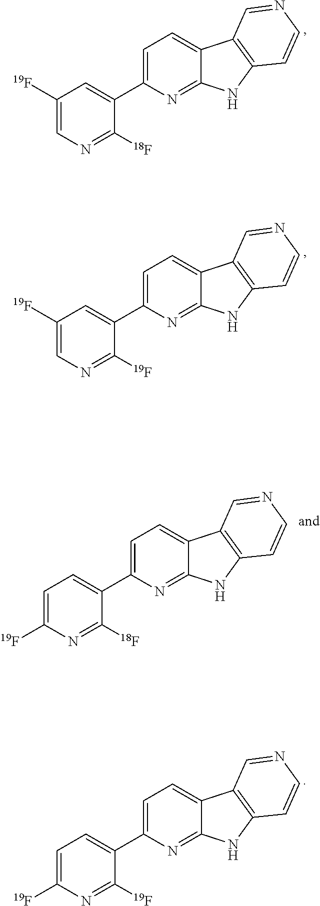 [SMILES: [18F]c1ncc([19F])cc1-c1ccc2c(n1)[nH]c1ccncc12 ; [19F]c1cnc([19F])c(-c2ccc3c(n2)[nH]c2ccncc23)c1 ; [18F]c1nc([19F])ccc1-c1ccc2c(n1)[nH]c1ccncc12 ; [19F]c1ccc(-c2ccc3c(n2)[nH]c2ccncc23)c([19F])n1]

16. The compound according to item 14, wherein
   R¹ is ¹⁸F, and
   R² is as defined in item 4.
17. The compound according to item 1, wherein the compound has the formula (III)

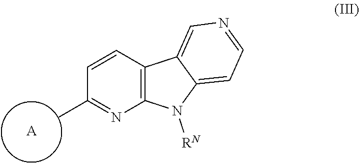

wherein

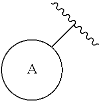

is selected from

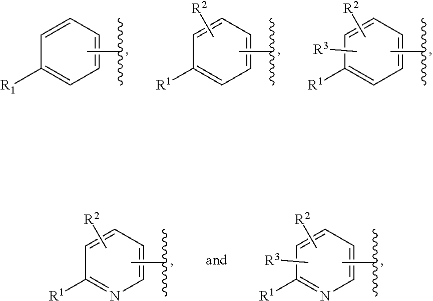

R¹ is LG,
R² and R³ are independently selected from the group consisting of halogen, alkyl, alkoxy, —NR⁷R⁸, —N(R⁷)alkyl, —N(alkyl)₂, and cyano, preferably R² and R³ are independently selected from the group consisting of halogen, —NR⁷R⁸, —N(R⁷)alkyl, —N(alkyl)₂, and cyano, more preferably R² and R³ are independently selected from the group consisting of halogen and cyano,
wherein the alkyl group(s) in alkyl, alkoxy, —N(R⁷)alkyl and —N(alkyl)₂ are independently optionally substituted with one or more halogen(s),
R⁷ and R⁸ are independently selected from the group consisting of hydrogen and PG1,
LG is a leaving group,
R^{N} is selected from the group consisting of hydrogen and PG2, and
PG1 and PG2 are independently selected from amine protecting groups.
18. The compound according to item 17, wherein

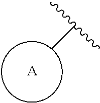

is selected from

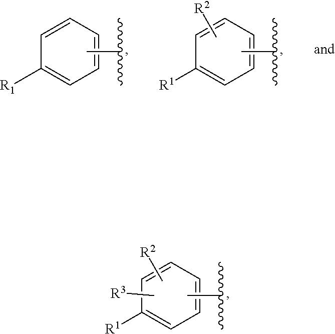

and
R¹, R², and R³ are as defined in item 17.
19. The compound according to item 18, wherein the compound is selected from

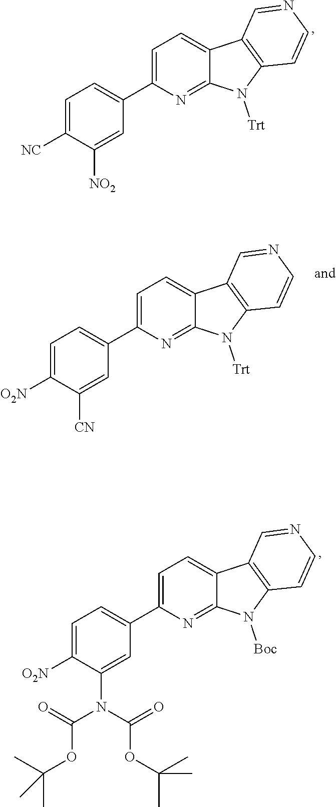

wherein
Boc is butyloxycarbonyl, and
Trt is triphenylmethyl.
20. The compound according to item 17, wherein

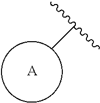

is selected from

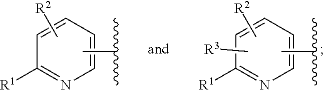

and
R¹, R², and R³ are as defined in item 17.
21. The compound according to item 20, wherein

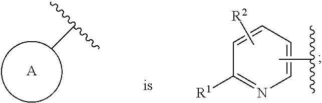

and
R¹ and R² are as defined in item 17.
22. The compound according to item 17, wherein the compound has the formula (IIIa)

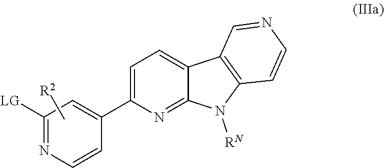

wherein LG, R² and R^{N} are as defined in item 17.
23. The compound according to item 22, wherein the compound is selected from

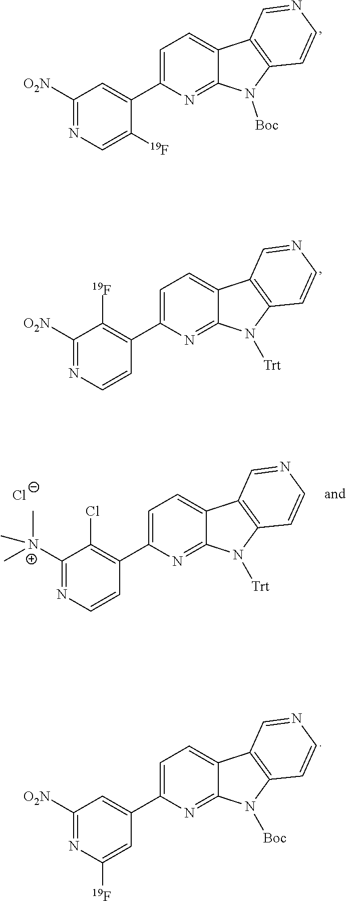

wherein
Boc is butyloxycarbonyl, and
Trt is triphenylmethyl.
24. The compound according to item 17, wherein the compound has the formula (IIIb)

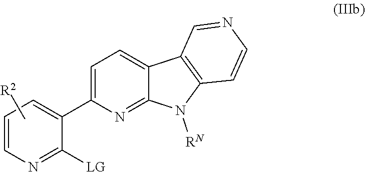

wherein LG, R² and R^{N} are as defined in item 17.
25. The compound according to item 24, wherein the compound is selected from

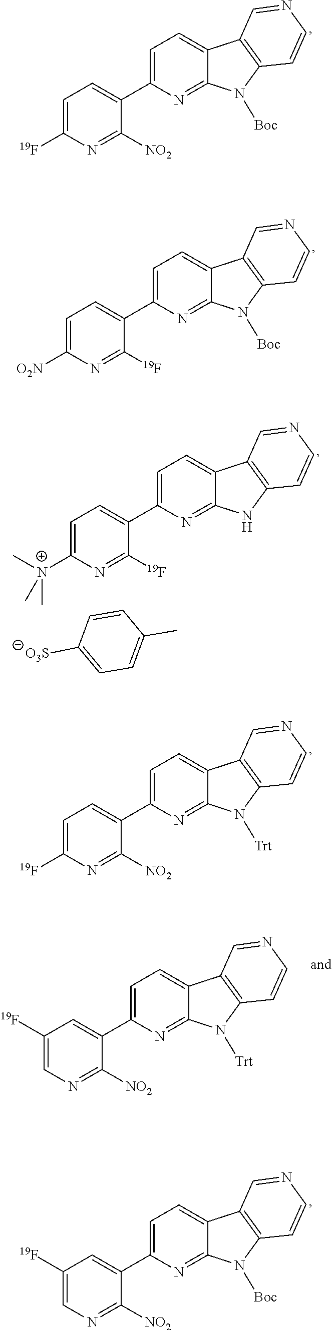

wherein
Boc is butyloxycarbonyl, and
Trt is triphenylmethyl.
26. The compound according to any of items 1 to 3, 17, 18, 20 to 22 and 24, wherein PG1 is a carbamate amine protecting group.
27. The compound according to any of items 1, 2, 3, 17, 18, 20, 21, 22, 24, and 26, wherein LG is selected from the group consisting of nitro, halogen and trimethyl ammonium.
28. The compound according to item 27, wherein LG is nitro.
29. The compound according to any of items 1, 2, 3, 17, 18, 20, 21, 22, 24, and 26, wherein PG2 is selected from the group consisting of tert-butyloxycarbonyl (BOC), triphenylmethyl (Trityl) and dimethoxytrityl (DMT).
30. The compound according to item 29, wherein PG2 is tert-butyloxycarbonyl (BOC).
31. The compound according to any of items 1 to 16, wherein the compound is detectably labeled.
32. The compound according to item 31, wherein the detectable label is selected from ²H, ³H and ¹⁸F.
33. The compound according to item 32, wherein the detectable label is ¹⁸F.
34. The compound according to any of items 1 to 16 and 31 to 33, wherein the compound contains no more than one ¹⁸F.
35. A diagnostic composition comprising a compound as defined in any of items 1 to 16 and 31 to 34 and optionally a pharmaceutically acceptable carrier, diluent, adjuvant or excipient.
36. A compound as defined in any of items 1 to 16 and 31 to 34 for use in diagnostics.
37. A compound as defined in any of items 1 to 16 and 31 to 34 for use in the imaging of Tau aggregates, particularly for use in positron emission tomography imaging of Tau aggregates.
38. A compound as defined in any of items 1 to 16 and 31 to 34 for use in the diagnosis of a disorder associated with Tau aggregates or for use in the diagnosis of a tauopathy, particularly wherein the diagnosis is conducted by positron emission tomography.
39. A compound for use according to item 38, wherein the tauopathy is a 3R tauopathy.
40. A compound for use according to item 38, wherein the tauopathy is a 4R tauopathy.
41. A compound for use according to item 38, wherein the disorder is selected from Alzheimer's disease (AD), familial AD, Creutzfeldt-Jacob disease, dementia pugilistica, Down's Syndrome, Gerstmann-Strsussler-Scheinker disease, inclusion-body myositis, prion protein cerebral amyloid angiopathy, traumatic brain injury (TBI), amyotrophic lateral sclerosis, Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease (PiD), progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, and lattice dystrophy.
42. A compound for use according to item 38, wherein the disorder is selected from Huntington's disease, ischemic stroke and psychosis in AD.
43. A compound for use according to item 41, wherein the disorder is Alzheimer's disease (AD).
44. A compound for use according to item 41, wherein the disorder is Parkinson's disease or atypical parkinsonism.
45. A compound for use according to item 41, wherein the disorder is progressive supranuclear palsy (PSP).
46. A compound for use according to item 41, wherein the disorder is Pick's disease (PiD).
47. The compound for use according to any of items 37 to 46, wherein the Tau aggregates are imaged in the brain or in the eye, preferably wherein the detectable label is ¹⁸F and the imaging is positron emission tomography.
48. A method of imaging of Tau aggregates, particularly a method of positron emission tomography imaging of Tau aggregates, wherein an effective amount of a compound as defined in any of items 1 to 16 and 31 to 34 is administered to a patient.
49. A method of diagnosing a disorder associated with Tau aggregates or a tauopathy, wherein an effective amount of a compound as defined in any of items 1 to 16 and 31 to 34 is administered to a patient, particularly wherein the diagnosis is conducted by positron emission tomography.
50. A method according to item 49, wherein the tauopathy is a 3R tauopathy.
51. A method according to item 49, wherein the tauopathy is a 4R tauopathy.
52. The method according to item 49, wherein the disorder is selected from Alzheimer's disease (AD), familial AD, Creutzfeldt-Jacob disease, dementia pugilistica, Down's Syndrome, Gerstmann-Strsussler-Scheinker disease, inclusion-body myositis, prion protein cerebral amyloid angiopathy, traumatic brain injury (TBI), amyotrophic lateral sclerosis, Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease (PiD), progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, and lattice dystrophy.
53. The method according to item 49, wherein the disorder is selected from Huntington's disease, ischemic stroke and psychosis in AD.
54. The method according to item 52, wherein the disorder is Alzheimer's disease (AD).
55. The method according to item 52, wherein the disorder is Parkinson's disease or atypical parkinsonism.
56. The method according to item 52, wherein the disorder is progressive supranuclear palsy (PSP).
57. The method according to item 52, wherein the disorder is Pick's disease (PiD).
58. The method according to any of items 48 to 57, wherein the Tau aggregates are imaged in the brain or in the eye, preferably wherein the detectable label is ¹⁸F and the imaging is positron emission tomography.
59. Use of the compound according to any of items 1 to 16 and 31 to 34 as an analytical reference.
60. Use of the compound according to any of items 1 to 16 and 31 to 34 as an in vitro screening tool.
61. A method of preparing a compound as defined in item 4 comprising reacting a compound as defined in item 17 with a [¹⁸F]fluorinating agent, wherein the method further comprises cleaving of the protecting group PG1 and/or PG2, if present.
62. The method according to item 61, wherein the [¹⁸F]fluorinating agent is selected from K¹⁸F, H¹⁸F, Cs¹⁸F, Na¹⁸F and a tetra(C₁₋₆ alkyl) ammonium salt of ¹⁸F.
63. A kit for preparing a radiopharmaceutical preparation, said kit comprising a sealed vial containing a predetermined quantity of a compound as defined in item 17.
64. The kit according to item 63, which further comprises at least one component selected from a reaction solvent, a solid-phase extraction cartridge, a reagent for cleaving the protecting group, a solvent for purification, a solvent for formulation and a pharmaceutically acceptable carrier, diluent, adjuvant or excipient for formulation.
65. A method of collecting data for the diagnosis of a disorder associated with tau aggregates in a sample or a patient comprising:
   (a) bringing a sample or a specific body part or body area suspected to contain a tau aggregate into contact with a compound as defined in any of items 1 to 16 and 31 to 34;
   (b) allowing the compound to bind to the tau aggregate;
   (c) detecting the compound bound to the tau aggregate; and
   (d) optionally correlating the presence or absence of compound binding with the tau aggregate with the presence or absence of tau aggregate in the sample or specific body part or body area.
66. A method of collecting data for determining a predisposition to a disorder associated with tau aggregates in a patient comprising detecting the specific binding of a compound as defined in any of items 1 to 16 and 31 to 34 to a tau aggregate in a sample or in situ which comprises the steps of:
   (a) bringing the sample or a specific body part or body area suspected to contain the tau aggregate into contact with the compound as defined in any of items 1 to 16 and 31 to 34, which compound specifically binds to the tau aggregate;
   (b) allowing the compound to bind to the tau aggregate to form a compound/tau aggregate complex;
   (c) detecting the formation of the compound/tau aggregate complex;
   (d) optionally correlating the presence or absence of the compound/tau aggregate complex with the presence or absence of tau aggregate in the sample or specific body part or body area; and
   (e) optionally comparing the amount of the compound/tau aggregate to a normal control value.
67. A method of collecting data for predicting responsiveness of a patient suffering from a disorder associated with tau aggregates and being treated with a medicament comprising:
   (a) bringing a sample or a specific body part or body area suspected to contain an tau aggregate into contact with a compound as defined in any of items 1 to 16 and 31 to 34, which compound specifically binds to the tau aggregate;
   (b) allowing the compound to bind to the tau aggregate to form a compound/tau aggregate complex;
   (c) detecting the formation of the compound/tau aggregate complex;
   (d) optionally correlating the presence or absence of the compound/tau aggregate complex with the presence or absence of tau aggregate in the sample or specific body part or body area; and
   (e) optionally comparing the amount of the compound/tau aggregate to a normal control value.

It is understood that the present invention covers compounds of the formula (I) in which one or more of the respective atoms is replaced by a different isotope. For instance, the compounds of the formula (I) include compounds in which one or more of the hydrogen atoms is replaced by tritium and/or one or more of the hydrogen atoms is replaced by deuterium.

In particular, is understood that the present invention covers compounds of the formula (II) in which one or more of the respective atoms is replaced by a different isotope. For instance, the compounds of the formula (II) include compounds in which one or more of the hydrogen atoms is replaced by tritium and/or one or more of the hydrogen atoms is replaced by deuterium.

The present inventors have surprisingly found that the exemplified compounds of the formula (II) have significantly improved properties compared to the prior art compounds ¹⁸F-1 or ¹⁸F-2.

Examples thereof include:

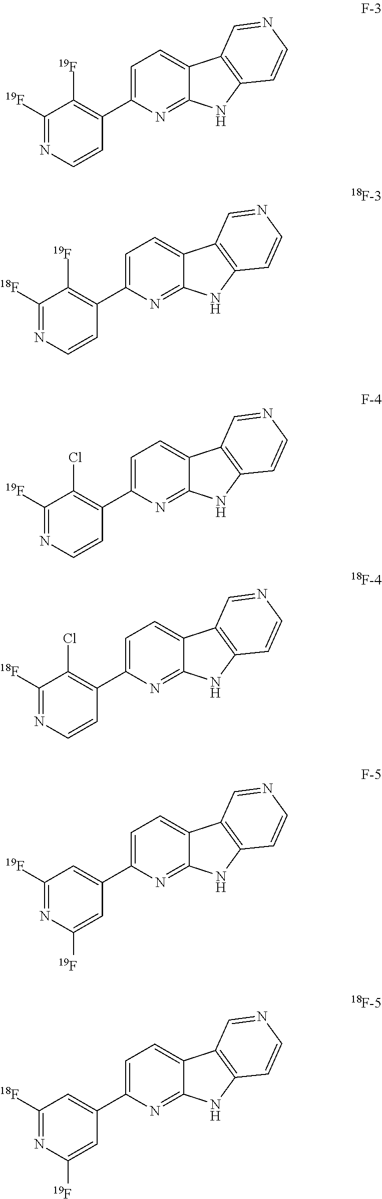 [SMILES: [19F]c1nccc(-c2ccc3c(n2)[nH]c2ccncc23)c1[19F] ; [18F]c1nccc(-c2ccc3c(n2)[nH]c2ccncc23)c1[19F] ; [19F]c1nccc(-c2ccc3c(n2)[nH]c2ccncc23)c1Cl ; [18F]c1nccc(-c2ccc3c(n2)[nH]c2ccncc23)c1Cl ; [19F]c1cc(-c2ccc3c(n2)[nH]c2ccncc23)cc([19F])n1 ; [18F]c1cc(-c2ccc3c(n2)[nH]c2ccncc23)cc([19F])n1]

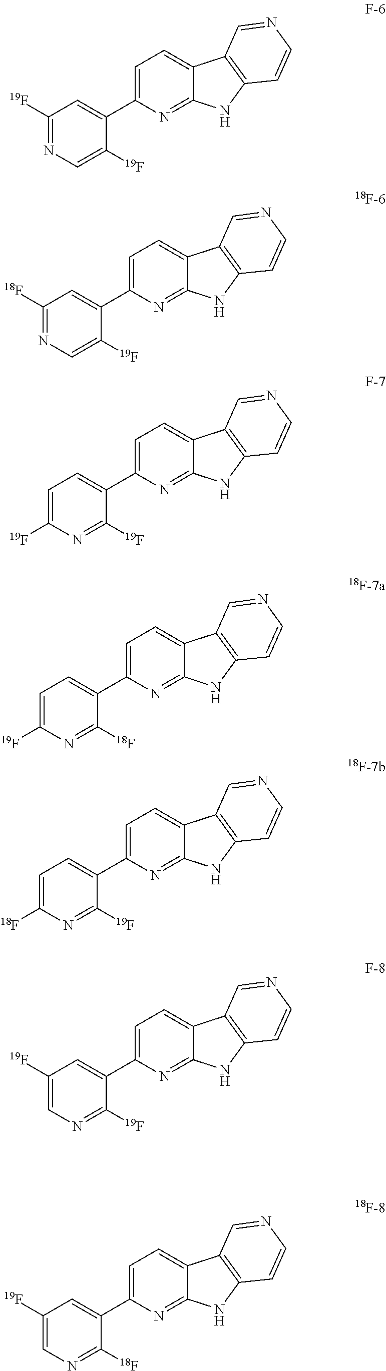

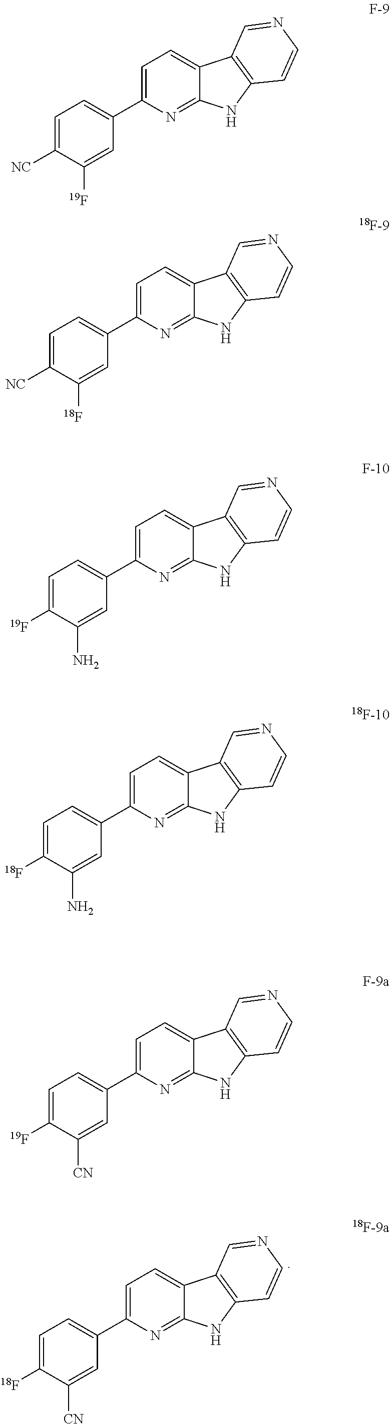 [SMILES: N#Cc1ccc(-c2ccc3c(n2)[nH]c2ccncc23)cc1[19F] ; N#Cc1ccc(-c2ccc3c(n2)[nH]c2ccncc23)cc1[18F] ; Nc1cc(-c2ccc3c(n2)[nH]c2ccncc23)ccc1[19F] ; Nc1cc(-c2ccc3c(n2)[nH]c2ccncc23)ccc1[18F] ; N#Cc1cc(-c2ccc3c(n2)[nH]c2ccncc23)ccc1[19F] ; N#Cc1cc(-c2ccc3c(n2)[nH]c2ccncc23)ccc1[18F]]

### DETAILED DESCRIPTION

The present invention relates to compounds of the formula (I)

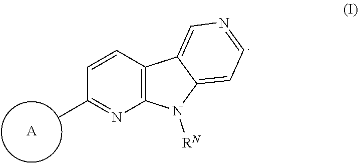

In particular, the present invention provides compounds of the formula (II)

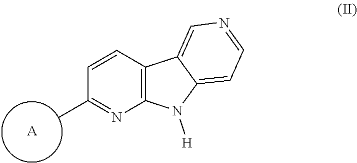

which are suitable in diagnosis. These compounds can be prepared from intermediates of the formula (III)

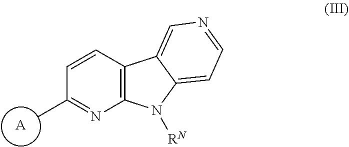

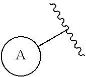

is selected from

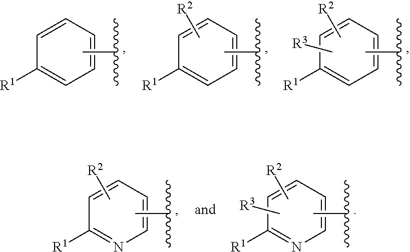

In a preferred embodiment,

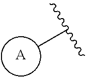

is selected from

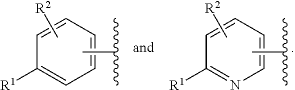

In another embodiment,

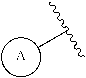

is selected from

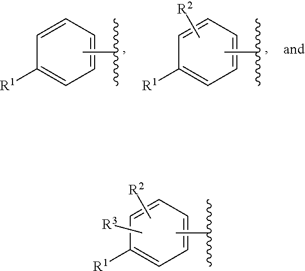

In yet another embodiment,

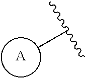

is selected from

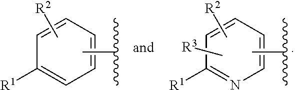

In a further embodiment,

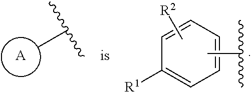

In yet a further embodiment,

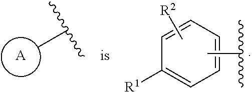

R¹ is selected from the group consisting of ¹⁸F, ¹⁹F and LG. In a preferred embodiment, R¹ is ¹⁸F or ¹⁹F, more preferably R¹ is ¹⁸F. In another embodiment, R¹ is LG.

R² is selected from the group consisting of halogen, alkyl, alkoxy, —NR⁷R⁸, —N(R)alkyl, —N(alkyl)₂, and cyano. In a preferred embodiment, R² is selected from the group consisting of halogen, —NR⁷R⁸, —N(R)alkyl, —N(alkyl)₂, and cyano. More preferably, R² is selected from the group consisting of halogen, —NH₂, —N(H)alkyl, —N(alkyl)₂, and cyano. Even more preferably, R² is selected from the group consisting of halogen and cyano. In a most preferable embodiment, R² is halogen, particularly F or Cl. In another preferred embodiment, R² is —NR⁷R⁸ or —N(R)alkyl with R¹ being PG1. It is to be understood that the alkyl group(s) in alkyl, alkoxy, —N(H)alkyl, —N(R⁷)alkyl and —N(alkyl)₂ are independently optionally substituted with one or more halogen(s).

R³ is selected from the group consisting of halogen, alkyl, alkoxy, —NR⁷R⁸, —N(R⁷)alkyl, —N(alkyl)₂, and cyano. In a preferred embodiment, R³ is selected from the group consisting of halogen, —NR⁷R⁸, —N(R⁷)alkyl, —N(alkyl)₂, and cyano. More preferably, R³ is selected from the group consisting of halogen, —NH₂, —N(H)alkyl, —N(alkyl)₂, and cyano. Even more preferably, R³ is selected from the group consisting of halogen and cyano. In a most preferable embodiment, R³ is halogen, particularly F or Cl. In another preferred embodiment, R³ is —NR⁷R⁸ or —N(R⁷)alkyl with R⁷ being PG1. It is to be understood that the alkyl group(s) in alkyl, alkoxy, —N(H)alkyl, —N(R⁷)alkyl and —N(alkyl)₂ are independently optionally substituted with one or more halogen(s).

R⁷ is selected from the group consisting of hydrogen and PG1. In a preferred embodiment, R⁷ is hydrogen. In another preferred embodiment, R⁷ is PG1.

R⁸ is selected from the group consisting of hydrogen and PG1. In a preferred embodiment, R⁸ is hydrogen. In another preferred embodiment, R⁸ is PG1.

R^{N} is selected from the group consisting of hydrogen and PG2. In a preferred embodiment, R^{N} is hydrogen. In another preferred embodiment, R^{N} is PG2.

LG is a leaving group.

PG1 is selected from amine protecting groups.

PG2 is selected from amine protecting groups.

Combination of the above definitions and preferred definitions are also envisaged.

Preferred compounds of the present invention are compounds of formula (IIa) and (IIb)

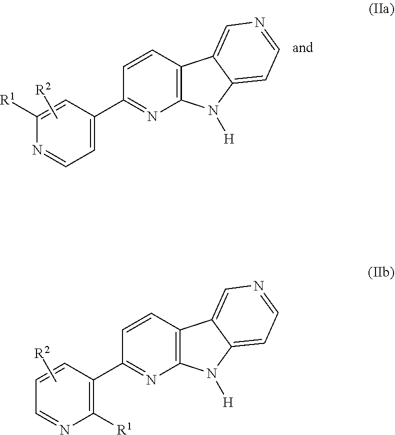

wherein R¹ and R² are as defined above.

Also preferred are the following compounds:

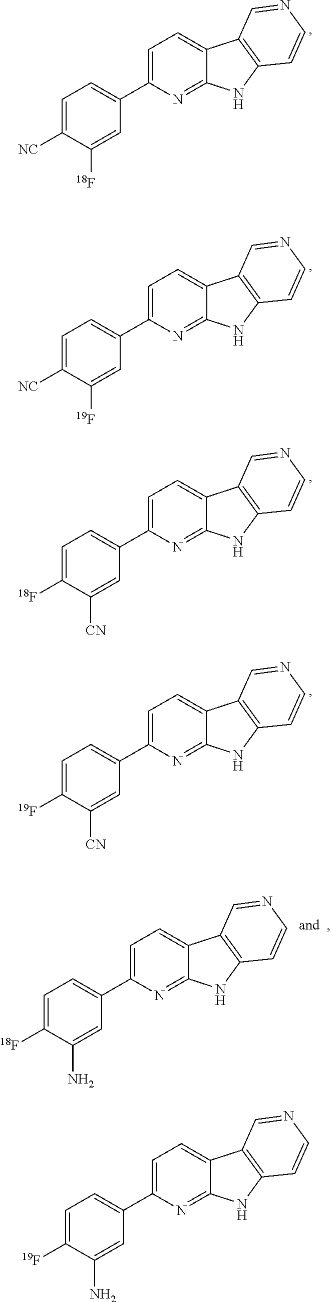 [SMILES: N#Cc1ccc(-c2ccc3c(n2)[nH]c2ccncc23)cc1[18F] ; N#Cc1ccc(-c2ccc3c(n2)[nH]c2ccncc23)cc1[19F] ; N#Cc1cc(-c2ccc3c(n2)[nH]c2ccncc23)ccc1[18F] ; N#Cc1cc(-c2ccc3c(n2)[nH]c2ccncc23)ccc1[19F] ; Nc1cc(-c2ccc3c(n2)[nH]c2ccncc23)ccc1[18F] ; Nc1cc(-c2ccc3c(n2)[nH]c2ccncc23)ccc1[19F]]

More preferred compounds of the present invention are

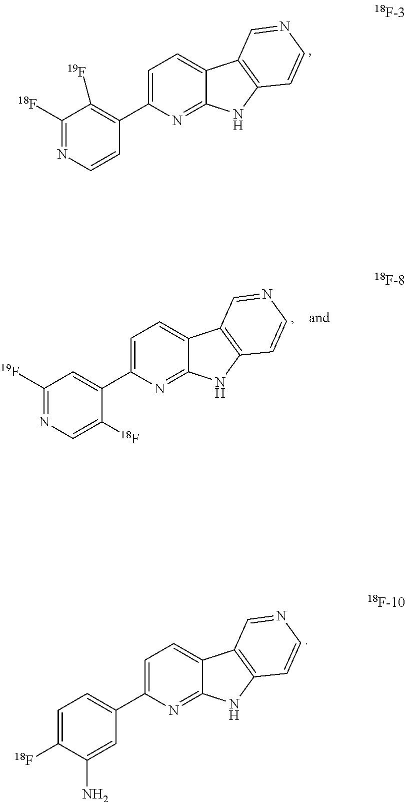 [SMILES: [18F]c1nccc(-c2ccc3c(n2)[nH]c2ccncc23)c1[19F] ; [18F]c1cnc([19F])cc1-c1ccc2c(n1)[nH]c1ccncc12 ; Nc1cc(-c2ccc3c(n2)[nH]c2ccncc23)ccc1[18F]]

An even more preferred compound of the present invention is

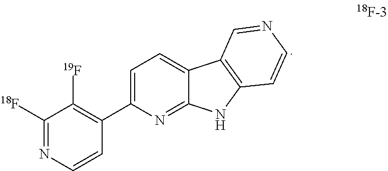 [SMILES: [18F]c1nccc(-c2ccc3c(n2)[nH]c2ccncc23)c1[19F]]

Another even more preferred compound of the present invention is

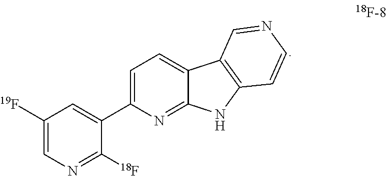 [SMILES: [18F]c1ncc([19F])cc1-c1ccc2c(n1)[nH]c1ccncc12]

Detectably labeled compounds of the present invention can be employed in the selective detection of disorders and abnormalities associated with Tau aggregates such as Alzheimer's disease and other tauopathies, for example, by using Positron Emission Tomography (PET) imaging.

The present invention also refers to intermediates which can be used in the production of such imaging compounds. The intermediates are compounds of the formula (III) as defined above.

The present compounds have a high affinity for Tau and/or bind to Tau-isoforms present in both, Alzheimer's disease (AD), as well as in non-AD tauopathies, such as for example progressive supranuclear palsy (PSP), and Pick's disease (PiD). Since they have a low affinity for amyloid-beta, MAO A and MAO B, they can be used as highly selective molecular probes for binding pathological Tau and thus avoid detection of other pathologies and misdiagnosis.

The instant ¹⁸F-labeled compounds also lead to a low signal in healthy brain, so that they can reduce background signal interference and thus provide a low detection limit.

Due to their good brain uptake, fast washout from healthy brain, low long-term retention in healthy brain as well as the lack of in vivo de-fluorination the instant ¹⁸F-labeled compounds provide a good signal-to-noise ratio.

Furthermore, the instant compounds can be easily detectably labeled, e.g., with ¹⁸F, in high yields.

### Definitions

The term “alkyl” refers to a saturated straight or branched carbon chain, which, unless specified otherwise, contain from 1 to 6 carbon atoms. The alkyl group can be optionally substituted with one or more halogen(s). The one or more halogen(s) are preferably selected from ¹⁹F and ¹⁸F.

The term “alkoxy” refers to an —O-alkyl group.

“Hal” or “halogen” represents F, Cl, Br and I. Preferably, “halogen” is, independently in each occurrence, selected from F, Cl and Br, more preferably, from F and Cl, even more preferably F.

The term “amine protecting group” (PG1 or PG2) as employed herein is any protecting group which is suitable for protecting an amine group during an envisaged chemical reaction.

Examples of suitable protecting groups are well-known to a person skilled in the art. Suitable protecting groups are discussed, e.g., in the textbook Greene and Wuts, Protecting groups in Organic Synthesis, third edition, page 494-653, which is included herein by reference.

Protecting groups can be chosen from carbamates, amides, imides, N-alkyl amines, N-aryl amines, imines, enamines, boranes, N-P protecting groups, N-sulfenyl, N-sulfonyl and N-silyl. Specific preferred examples of amine protecting groups (PG1 or PG2) are carbobenzyloxy (Cbz), (p-methoxybenzyl)oxycarbonyl (Moz or MeOZ), tert-butyloxycarbonyl (BOC), 9-fluorenylmethyloxycarbonyl (FMOC), benzyl (Bn), p-methoxybenzyl (PMB), 3,4-dimethoxybenzyl (DMPM), p-methoxyphenyl (PMP), triphenylmethyl (Trityl), methoxyphenyl diphenylmethyl (MMT), or dimethoxytrityl (DMT). More preferred examples of the amine protecting group PG1 or PG2 include tert-butyloxycarbonyl (BOC), dimethoxytrityl (DMT) and triphenylmethyl (Trityl). One more preferred example of the amine protecting group PG1 or PG2 is tert-butyloxycarbonyl (BOC).

The term “carbamate amine protecting group” refers to an amine protecting group containing a *—CO—O group wherein the asterisk indicates the bond to the amine. Examples are carbobenzyloxy (Cbz), (p-methoxybenzyl)oxycarbonyl (Moz or MeOZ), tert-butyloxycarbonyl (BOC) and 9-fluorenylmethyloxycarbonyl (FMOC).

The term “leaving group” (LG) as employed herein is any leaving group and means an atom or group of atoms can be replaced by another atom or group of atoms. Examples are given e.g. in Synthesis (1982), p. 85-125, table 2, Carey and Sundberg, Organische Synthese, (1995), page 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33). Preferably, the “leaving group” (LG) is nitro, halogen or trimethyl ammonium. More preferably, “leaving group” (LG) is nitro.

Tau as used herein refers to a highly soluble microtubule binding protein mostly found in neurons and includes the major 6 isoforms, cleaved or truncated forms, and other modified forms such as arising from phosphorylation, glycosylation, glycation, prolyl isomerization, nitration, acetylation, polyamination, ubiquitination, sumoylation and oxidation. Pathologic Tau or Tau aggregates (Neurofibrillary Tangles, NFTs) as used herein refer to insoluble aggregates of the hyperphosphorylated Tau protein containing paired helical filaments and straight filaments. Their presence is a hallmark of AD and other diseases known as tauopathies.

The term “crown ether” as employed herein means chemical compounds that consist of a ring containing several ether groups. More specifically, the term “crown ether” refers to preferably monocyclic organic groups which may be substituted and contain from 8 to 16 carbon atoms and from 4 to 8 heteroatoms selected from N, O and S in the ring. Each of the one or more optional substituents may be independently selected from any organic group containing from 1 to 15 carbon atoms and optionally 1 to 6 heteroatoms selected from N, O and S. Preferred examples of the “crown ether” are optionally substituted monocyclic rings containing 10 to 14 carbon atoms and 5 to 7 heteroatoms selected from N, O and S in the ring. Examples of the “crown ether” are optionally substituted monocyclic rings containing 12 carbon atoms and 6 heteroatoms selected from N and O in the ring. Specific examples include 18-crown-6, dibenzo-18-crown-6, and diaza-18-crown-6.

The term “cryptand” as employed herein relates to a class of polycyclic compounds related to the crown ethers, having three chains attached at two nitrogen atoms. A well-known “cryptand” is 4,7,13,16,21,24-hexaoxa-1,10-diazabicyclo[8.8.8]hexacosane (Kryptofix®).

The tau gene contains 16 exons with the major tau protein isoforms being encoded by 11 of them The alternative splicing of exon 10 generates tau isoforms with either three (exon 10 missing) or four (exon 10 present) repeat domains, known as 3R and 4R tau, respectively (A. Andreadis et al., Biochemistry 31, (1992) 10626-10633; M. Tolnay et al., IUBMB Life, 55(6): 299-305, 2003). In Alzheimer's disease, the ratio of 3R and 4R isoforms is similar. In contrast thereto, in some tauopathies one of the two isoforms is predominantly present. Herein, the term “3R tauopathy” refers to tauopathies (such as Pick's disease (PiD)) in which the 3R isoform is predominantly present. Herein, the term “4R tauopathy” refers to tauopathies (such as progressive supranuclear palsy (PSP) and corticobasal degeneration (CBD)) in which the 4R isoform is predominantly present.

The term “polymorphs” refers to the various crystalline structures of the compounds of the present invention. This may include, but is not limited to, crystal morphologies (and amorphous materials) and all crystal lattice forms. Salts of the present invention can be crystalline and may exist as more than one polymorph.

Solvates, hydrates as well as anhydrous forms of the present compounds are also encompassed by the invention. The solvent included in the solvates is not particularly limited and can be any pharmaceutically acceptable solvent. Examples include water and C-alcohols (such as methanol or ethanol).

As used hereinafter in the description of the invention and in the claims, the term “prodrug” means any covalently bonded compound which releases the active parent pharmaceutical due to in vivo biotransformation. The reference by Goodman and Gilman (The Pharmacological Basis of Therapeutics, 8 ed, McGraw-Hill, Int. Ed. 1992, “Biotransformation of Drugs”, p 13-15) describing prodrugs generally is hereby incorporated herein by reference.

As used hereinafter in the description of the invention and in the claims, the term “pharmaceutically acceptable salt” relates to non-toxic derivatives of the disclosed compounds wherein the parent compound is modified by making salts of inorganic and organic acids thereof. Inorganic acids include, but are not limited to, acids such as carboxylic, hydrochloric, nitric or sulfuric acid. Organic acids include, but are not limited to, acids such as aliphatic, cycloaliphatic, aromatic, araliphatic, heterocyclic, carboxylic and sulphonic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts can be found in Remington's Pharmaceutical Sciences, 18th ed., Mack Publishing Company, Easton, Pa., 1990, p. 1445, the disclosure of which is hereby incorporated by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The patients or subjects in the present invention are typically animals, particularly mammals, more particularly humans.

The preferred definitions given in the “Definition”-section apply to all of the embodiments described herein unless stated otherwise.

### Diagnostic Procedures

The detectably labeled compounds of the formula (II) are particularly suitable for imaging of Tau protein aggregates. With respect to Tau protein, the detectably labeled compounds of the formula (II) are able to bind to various types of Tau aggregates such as pathologically aggregated Tau, hyperphosphorylated Tau, neurofibrillary tangles, paired helical filaments, straight filaments, neurotoxic soluble oligomers, polymers and fibrils.

Due to the above binding characteristics, the detectably labeled compounds of the formula (II) are suitable for use in the diagnosis of disorders associated with Tau aggregates. The detectably labeled compounds of the formula (II) are particularly suitable for positron emission tomography (PET) imaging of Tau deposits. Typically ¹⁸F labeled compounds of the formula (II) are employed as detectably labeled compounds if the compounds are to be administered to a patient.

In the imaging of Tau aggregates a detectably labeled compound of the formula (II) is administered and the signal stemming from the compound that is specifically bound to the Tau aggregates is detected. The specific binding is a result of the high binding affinity of the compounds of the formula (II) to the Tau aggregates.

In a preferred embodiment, a detectably labeled compound of the formula (II) is employed for diagnosing whether a tauopathy (preferably Alzheimer's disease) is present. In this method a detectably labeled compound of the formula (II) is administered to a patient who is suspected to suffer from a tauopathy (preferably Alzheimer's disease) or a sample obtained from such a patient and the signal stemming from the detectable label is detected, preferably by positron emission tomography (PET).

If no signal stemming from the detectable label is detected then the instant method can be used to exclude a tauopathy, which indicates that a neurological disorder other than a tauopathy is present.

In the methods of diagnosing a disorder associated with Tau protein aggregates such as Alzheimer's disease, or a predisposition therefor in a subject, the method comprising:
a) administering to the mammal a diagnostically effective amount of a detectably labeled compound of the formula (II);
b) allowing the detectably labeled compound of the formula (II) to distribute into the tissue of interest (such as brain tissue or body fluids such as cerebrospinal fluid (CSF)); and
c) imaging the tissue of interest, wherein an increase in binding of the detectably labeled compound of the formula (II) to the tissue of interest compared to a normal control level of binding indicates that the subject is suffering from or is at risk of developing a disorder associated with Tau protein aggregates.

The detectably labeled compounds of the formula (II) can be used for imaging of Tau protein aggregates in any sample or a specific body part or body area of a patient which suspected to contain a Tau protein aggregate. The detectably labeled compounds of the formula (II) are able to pass the blood-brain barrier. Consequently, they are particularly suitable for imaging of Tau protein aggregates in the brain, as well as in body fluids such as cerebrospinal fluid (CSF).

In diagnostic applications, the detectably labeled compounds of the formula (II) are preferably administered in a diagnostic composition.

Diagnosis of a Tau disorder or of a predisposition to a Tau-associated disorder in a patient may be achieved by detecting the specific binding of a detectably labeled compound of the formula (II) to the Tau protein aggregates in a sample or in situ, which includes:
(a) bringing the sample or a specific body part or body area suspected to contain the Tau protein aggregate into contact with a detectably labeled compound of the formula (II) which binds the Tau protein aggregate;
(b) allowing the detectably labeled compound of the formula (II) to bind to the Tau protein aggregate to form a compound/Tau protein aggregate complex (hereinafter “compound/Tau protein aggregate complex” will be abbreviated as “compound/protein aggregate complex”);
(c) detecting the formation of the compound/protein aggregate complex,
(d) optionally correlating the presence or absence of the compound/protein aggregate complex with the presence or absence of Tau protein aggregates in the sample or specific body part or area; and
(e) optionally comparing the amount of the compound/protein aggregate complex to a normal control value, wherein an increase in the amount of the compound/protein aggregate complex compared to a normal control value may indicate that the patient is suffering from or is at risk of developing a Tau-associated disorder.

After the sample or a specific body part or body area has been brought into contact with the detectably labeled compound of the formula (II), the compound is allowed to bind to the Tau protein aggregate. The amount of time required for binding will depend on the type of test (e.g., in vitro or in vivo) and can be determined by a person skilled in the field by routine experiments.

The compound which has bound to the Tau protein aggregate can be subsequently detected by any appropriate method. A preferred method is positron emission tomography (PET).

The presence or absence of the compound/protein aggregate complex is then optionally correlated with the presence or absence of Tau protein aggregates in the sample or specific body part or area. Finally, the amount of the compound/protein aggregate complex can be compared to a normal control value which has been determined in a sample or a specific body part or body area of a healthy subject, wherein an increase in the amount of the compound/protein aggregate complex compared to a normal control value may indicate that the patient is suffering from or is at risk of developing a Tau-associated disorder.

Predicting responsiveness of a patient suffering from a disorder associated with Tau protein aggregates and being treated with a medicament can be achieved by
(a) bringing a sample or a specific body part or body area suspected to contain a Tau protein aggregate into contact with a detectably labeled compound of the formula (II);
(b) allowing the detectably labeled compound of the formula (II) to bind to the Tau protein aggregate to form a compound/protein aggregate complex;
(c) detecting the formation of the compound/protein aggregate complex;
(d) optionally correlating the presence or absence of the compound/protein aggregate complex with the presence or absence of Tau protein aggregate in the sample or specific body part or body area; and
(e) optionally comparing the amount of the compound/protein aggregate complex to a normal control value.

How steps (a) to (e) can be conducted has already been explained above.

In the method for predicting responsiveness the amount of the compound/protein aggregate complex can be optionally compared at various points of time during the treatment, for instance, before and after onset of the treatment or at various points of time after the onset of the treatment. A change, especially a decrease, in the amount of the compound/protein aggregate complex may indicate that the patient has a high potential of being responsive to the respective treatment.

A compound according to the present invention can also be incorporated into a test kit for detecting a Tau protein aggregate. The test kit typically comprises a container holding one or more compounds according to the present invention and instructions for using the compound for the purpose of binding to a Tau protein aggregate to form a compound/protein aggregate complex and detecting the formation of the compound/protein aggregate complex such that presence or absence of the compound/protein aggregate complex correlates with the presence or absence of the Tau protein aggregates. In one embodiment, the test kit can contain a compound of the formula (II). In an alternative embodiment, the test kit can contain a compound of the formula (III) and a [¹⁸F]fluorinating agent, so that the compound of the formula (II) can be prepared shortly before the detection of the Tau protein aggregate is to take place.

The term “test kit” refers in general to any diagnostic kit known in the art. More specifically, the latter term refers to a diagnostic kit as described in Zrein et al., Clin. Diagn. Lab. Immunol., 1998, 5, 45-49.

### Diagnostic Compositions

A “diagnostic composition” is defined in the present invention as a composition comprising a detectably labeled compound of the formula (II) (preferably ¹⁸F labeled; in particular ¹⁸F-3, more particularly ¹⁸F-3a). For in vivo applications the diagnostic composition should be in a form suitable for administration to mammals such as humans. Preferably a diagnostic composition further comprises a physiologically acceptable carrier, diluent, adjuvant or excipient. Administration to a patient is preferably carried out by injection of the composition as an aqueous solution. Such a composition may optionally contain further ingredients such as solvents, buffers; pharmaceutically acceptable solubilizers; and pharmaceutically acceptable stabilizers or antioxidants.

Pharmaceutically acceptable excipients are well known in the pharmaceutical art, and are described, for example, in Remington's Pharmaceutical Sciences, 15^{th} Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

Pharmaceutically useful excipients that may be used in the formulation of the diagnostic composition of the present invention may comprise, for example, carriers, vehicles, diluents, solvents and edible oils, oily esters, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers.

If the detectably labeled compounds of the formula (II) (preferably ¹⁸F labeled, in particular ¹⁸F-3, more particularly ¹⁸F-3a) are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other excipients. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The dose of the detectably labeled compounds of the formula (II) will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

The diagnostic compositions of the invention can be produced in a manner known per se to the skilled person as described, for example, in Remington's Pharmaceutical Sciences, 15^{th} Ed., Mack Publishing Co., New Jersey (1975).

In particular, in one embodiment diseases or disorders that can be detected and monitored with the detectably labeled compounds of the formula (II) are diseases or conditions associated Tau proteins aggregates.

For instance, the compounds of the formula (II) can be employed in a liposomal composition as described in WO2016057812A1 which comprises a compound of formula (II) as a ligand for use in the selective detection of disorders and abnormalities associated with Tau aggregates by nonradioactive magnetic resonance imaging (MRI).

The diseases or conditions that can be detected and monitored with the detectably labeled compounds of the present invention include neurodegenerative disorders such as tauopathies. Examples of diseases and conditions which can be detected and monitored are caused by or associated with the formation of neurofibrillary lesions. This is the predominant brain pathology in tauopathy. The diseases and conditions comprise a heterogeneous group of neurodegenerative diseases or conditions including diseases or conditions which show co-existence of Tau and amyloid pathologies. Examples of diseases involving Tau aggregates are generally listed as tauopathies and these include, but are not limited to, Alzheimer's disease (AD), Creutzfeldt-Jacob disease, dementia pugilistica, Down's Syndrome, Gerstmann-Strsussler-Scheinker disease, inclusion-body myositis, prion protein cerebral amyloid angiopathy, traumatic brain injury, amyotrophic lateral sclerosis, Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease (PiD), progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, and lattice dystrophy. Preferably the diseases and conditions which can be detected and monitored include Alzheimer's disease (AD), familial AD, Creutzfeldt-Jacob disease, dementia pugilistica, Down's Syndrome, Gerstmann-Strsussler-Scheinker disease, inclusion-body myositis, prion protein cerebral amyloid angiopathy, traumatic brain injury (TBI), amyotrophic lateral sclerosis, Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease (PiD), progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, and white matter tauopathy with globular glial inclusions, more preferably Alzheimer's disease (AD), Creutzfeldt-Jacob disease, dementia pugilistica, amyotrophic lateral sclerosis, argyrophilic grain disease, corticobasal degeneration (CBD), frontotemporal dementia with Parkinsonism linked to chromosome 17, Pick's disease (PiD), progressive supranuclear palsy (PSP), tangle only dementia, Parkinson dementia complex of Guam, Hallervorden-Spatz disease and fronto-temporal lobar degeneration. Preferably the disease or condition is Alzheimer's disease.

Further diseases or conditions that can be detected and monitored with the detectably labeled compounds of the present invention include Huntington's disease, ischemic stroke and psychosis in AD.

### General Synthesis of ¹⁸F-Labeled Compounds of the Formula (II)

Compounds having the formula (II) which are labeled by ¹⁸F can be prepared by reacting a compound of formula (III), in which R¹ is LG and R^{N} is hydrogen or PG2, with an ¹⁸F-fluorinating agent, so that the leaving group LG is replaced by ¹⁸F. The preparation includes the cleavage of the protecting group PG2, if present.

Any suitable ¹⁸F-fluorinating agent can be employed. Typical examples include H¹⁸F, alkali or alkaline earth ¹⁸F-fluorides (e.g., K¹⁸F, Rb¹⁸F, Cs¹⁸F, and Na¹⁸F). Optionally, the ¹⁸F-fluorination agent can be used in combination with a chelating agent such as a cryptand (e.g.: 4,7,13,16,21,24-hexaoxa-1,10-diazabicyclo[8.8.8]-hexacosane—Kryptofix®) or a crown ether (e.g.: 18-crown-6). Alternatively, the ¹⁸F-fluorinating agent can be a tetraalkyl ammonium salt of ¹⁸F or a tetraalkyl phosphonium salt of ¹⁸F; e.g., tetra(C₁₋₆ alkyl)ammonium salt of ¹⁸F or a tetra(C₁₋₆ alkyl)phosphonium salt of ¹⁸F. Examples thereof include tetrabutyl ammonium [¹⁸F]fluoride and tetrabutyl phosphonium [¹⁸F]fluoride. Preferably, the ¹⁸F-fluorination agent is K¹⁸F, H¹⁸F, Cs¹⁸F, Na¹⁸F or tetrabutyl ammonium [¹⁸F]fluoride.

The reagents, solvents and conditions which can be used for the ¹⁸F-fluorination are well-known to a person skilled in the field (L. Cai, S. Lu, V. Pike, Eur. J. Org. Chem 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile or DMSO.

If desired, the compound having the formula (III) can have R^{N} in the meaning of PG2, wherein the protecting group PG2 protects the amine during the ¹⁸F-fluorination reaction.

This amine protecting group can be subsequently removed. Methods for removing the amine protecting group are known in the art and include, but are not limited to, acidic cleavage.

If desired, the compound of formula (II) can be isolated and/or purified further before use. Corresponding procedures are well-known in the art.

The precursor compounds having the formula (III) in which R¹ is LG and R^{N} is hydrogen or PG2 can be provided in a kit which is suitable for producing the compounds of the formula (II) by reaction with a ¹⁸F-fluorinating agent. In one embodiment the kit comprises a sealed vial containing a predetermined quantity of the precursor compound of the formula (III). For instance, the kit can contain 1.5 to 75 μmol, preferably 7.5 to 50 μmol, more preferably 10 to 30 μmol of a precursor compound of the formula (III). Optionally, the kit can contain further components, such as a reaction solvent, a solid-phase extraction cartridge, a reagent to obtain the ¹⁸F-fluorinating agent, a reagent for cleaving the protecting group, a solvent for purification, a solvent for formulation and a pharmaceutically acceptable carrier, diluent, adjuvant or excipient for formulation.

The compounds of the formula (II) in which R¹ is ¹⁹F can be used as an analytical reference or an in vitro screening tool.

The compounds of the formula (II) in which R¹ is ¹⁹F can be used as an analytical reference for the quality control and release of a compound of the formula (II) in which R¹ is ¹⁸F and R^{N} is hydrogen.

The compounds of formula (II) in which R¹ is ¹⁹F can be used as an in vitro screening tool for characterization of tissue with Tau pathology and for testing of compounds targeting Tau pathology on such tissue.

The present invention illustrated by the following examples which should not be construed as limiting.

### EXAMPLES

All reagents and solvents were obtained from commercial sources and used without further purification. Proton (¹H) spectra were recorded on a Bruker DRX-400 MHz NMR spectrometer or on a Bruker AV-400 MHz NMR spectrometer in deuterated solvents. Mass spectra (MS) were recorded on an Advion CMS mass spectrometer. Chromatography was performed using silica gel (Fluka: Silica gel 60, 0.063-0.2 mm) and suitable solvents as indicated in the specific examples. Flash purification was conducted with a Biotage Isolera One flash purification system using HP-Sil (Biotage) or puriFlash-columns (Interchim) and the solvent gradient indicated in the specific examples. Thin layer chromatography (TLC) was carried out on silica gel plates with UV detection.

Although some of the present examples do not indicate that the respective compounds were detectably labeled, it is understood that corresponding detectably labeled compounds can be easily prepared, e.g., by using detectably labeled starting materials, such as starting materials containing ³H atoms.

Unless explicitly stated otherwise, “F” in the structures of the following examples refers to “¹⁹F”.

Abbreviations

| | |
|---|---|
| AD | Alzheimer's disease |
| BSA | bovine serum albumin |
| Boc, BOC | tert-butyloxycarbonyl |
| CBD | corticobasal degeneration |
| d.c. | corrected for decay |
| d | doublet |
| dd | doublet of doublet |
| ddd | doublet of doublet of doublet |
| dt | doublet of triplet |
| DMA | dimethylacetamide |
| DMF | N,N-dimethyl formamide |
| DMSO | dimethylsulfoxide |
| EI | electron ionisation |
| ELSD | evaporative light scattering detector |
| ESI | electrospray ionisation |
| FTD | Frontotemporal dementia |
| HPLC | high performance liquid chromatography |
| HC | Healthy control |
| GBq | Gigabequerel |
| K₂₂₂ | 4,7,13,16,21,24-hexaoxa-1,10-diazabicyclo[8.8.8]- |
| | hexacosane (Kryptofix 222) |
| MBq | Megabequerel |
| MS | mass spectrometry |
| MeCN | acetonitrile |
| m | multiplet |
| mc | centered multiplet |
| n.c.a. | non-carrier-added |
| n.d.c. | not decay corrected |
| NMR | nuclear magnetic resonance spectroscopy: chemical shifts (δ) |
| | are given in ppm. |
| PBS | phosphate-buffered saline |
| PET | Positron-Emission-Tomography |
| PiD | Pick's disease |
| PSP | progressive supranuclear palsy |
| q | quadruplet (quartet) |
| RT | room temperature |
| s | singulet |
| t | triplet |
| Tau | Tau protein, Tau deposits, Tau aggregates |
| TBI | Traumatic brain injury |
| Trt | trityl (triphenylmethyl) |
| TLC | thin layer chromatography |

### Preparative Example A

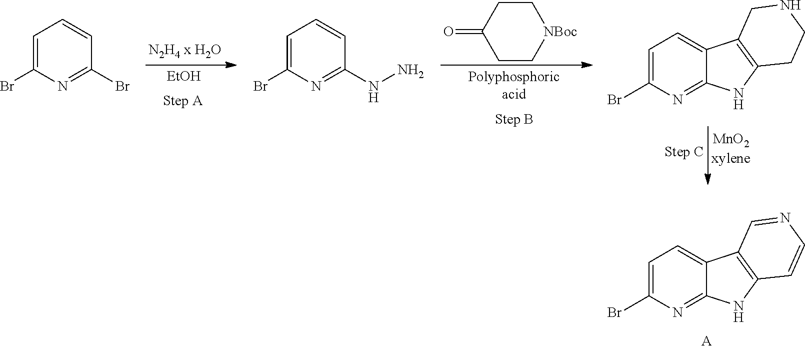

### Step A

Commercially available 2,6-dibromopyridine (4.12 g, 16.6 mmol) was suspended in ethanol (40 mL) and hydrazine hydrate (10 mL, 97.6 mmol) in water (˜50-60%) was added. The mixture was heated in a sand-bath at ˜115° C. for 18 hours. The solvent was removed and the residue was purified by chromatography on silica using ethyl acetate/n-heptane (60/40) to afford the title compound as an off-white solid (3.05 g, 93%).

¹H-NMR (400 MHz, CDCl₃): δ=7.33 (t, 1H), 6.83 (d, 1H), 6.67 (d, 1H), 6.00 (br-s, 1H), 3.33-3.00 (br-s, 2H)

### Step B

The title compound from Step A above (10 g, 53.2 mmol) and commercially available 1-Boc-4-piperidone (10.6 g, 53.2 mmol) were added to a 500 mL flask and mixed to become a homogenous blend. Then polyphosphoric acid (80 g, 115% H₃PO₄ basis) was added and the mixture was heated at 160° C. in a sand-bath. At 120° C. the Boc-protecting group was cleaved resulting in foaming of the reaction mixture. After complete Boc-cleavage the foam collapsed and the dark reaction mixture was stirred at 160° C. for 20 hours. The reaction mixture was allowed to cool to room temperature and water (400 mL) was added. The reaction mixture was stirred/sonicated until the gummy material was dissolved. The reaction mixture was then placed in an ice-bath and the pH of the solution was adjusted to pH ˜12 by adding solid sodium hydroxide pellets (exothermic). The precipitate was collected by filtration and washed with water (400 mL) to remove salts. The precipitate was dissolved in dichloromethane/methanol (9/1; 1500 mL) by sonication and washed with water (2×400 mL) to remove remaining salts and insoluble material. The organic phase was dried over Na₂SO₄, filtered and the solvents were removed under reduced pressure. The dark residue was treated with dichloromethane (100 mL), sonicated for 5 minutes and the precipitate was collected by filtration. The precipitate was washed with dichloromethane (40 mL) and air-dried to afford the title compound as a beige solid (3.5 g, 26%).

¹H-NMR (400 MHz, DMSO-d₆): δ=11.5 (br-s, 1H), 7.72 (d, 1H), 7.15 (d, 1H), 3.86-3.82 (m, 2H), 3.06-3.00 (m, 2H), 2.71-2.65 (m, 2H)

### Step C

The title compound from Step B above (1.75 g, 6.94 mmol) was suspended in xylene (380 mL) and manganese (IV) oxide (6.62 g, 76.9 mmol) was added. The reaction mixture was then heated at 160° C. in a sand-bath for 36 hours. The cooled reaction mixture was evaporated under reduced pressure, the residue was suspended in dichloromethane/methanol (1/1; 400 mL) and stirred at room temperature for 30 minutes. The reaction mixture was then filtered through paper filters to remove the manganese (IV) oxide and the filter was washed with methanol (50 mL). The combined filtrates were evaporated under reduced pressure and the dark residue was purified by chromatography on silica (50 g HP-SIL-cartridge) using a Biotage Isolera system employing an ethyl acetate/heptane gradient (5/95-100/0) to remove unpolar impurities followed by dichloromethane/methanol (9/1->4/1) to afford the title compound as a dark yellow solid. The total yield from 2 runs was 1.77 g (51%).

¹H-NMR (400 MHz, DMSO-d₆): δ=12.52 (br-s, 1H), 9.42 (s, 1H), 8.61 (d, 1H), 8.53 (d, 1H), 7.56-7.52 (m, 2H)

### Preparative Example B

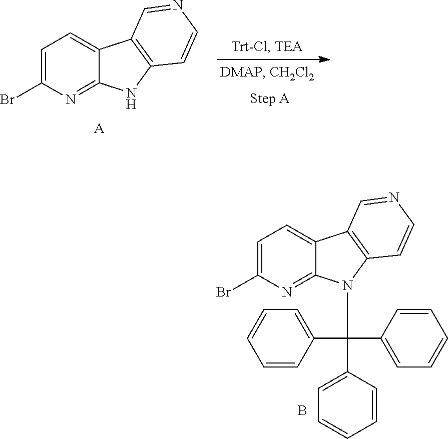

### Step A

To a suspension of the title compound from Preparative Example A (0.776 g, 0.72 mmol) in dichloromethane (65 mL) was added triethylamine (1.86 mL, 13 mmol) and trityl-chloride (2.63 g, 9.39 mmol). After the addition of 4-(dimethylamino)-pyridine (0.074 g, 0.608 mmol), the reaction mixture was stirred at room temperature for 16 hours. The reaction mixture was diluted with dichloromethane (150 mL) and water (50 mL). The organic phase was separated, dried over Na₂SO₄, filtered and the solvents were removed in vacuo. The residue was purified on HP-Sil SNAP cartridges (50 g) using a Biotage Isolera One purification system employing an ethyl acetate/n-heptane gradient (5/95->100/0->100/0) to afford the title compound B as a pale yellow solid (0.831 g, 54%). Unreacted starting material was recovered by flushing the cartridge with ethyl acetate/methanol (90/10) to afford the starting material as an off-white solid (0.195 g, 25%).

¹H-NMR (400 MHz, CDCl₃) δ=9.22 (s, 1H), 8.23 (d, 1H), 8.13 (d, 1H), 7.48-7.42 (m, 7H), 7.33-7.22 (m, 12H), 6.41 (d, 1H)

MS (ESI); m/z=490.03/491.96 [M+H]⁺

### Preparative Example C

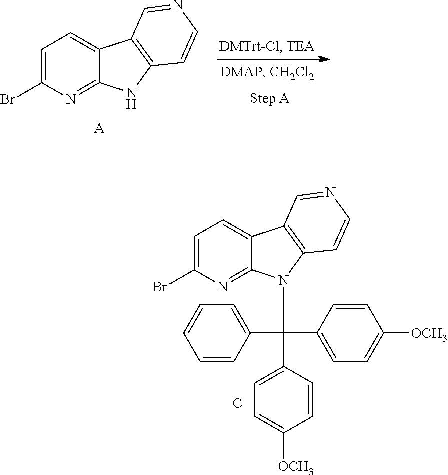

### Step A

To a suspension of the title compound from Preparative Example A (0.482 g, 1.94 mmol) in dichloromethane (40 mL) was added triethylamine (1.15 mL, 8 mmol) and 4,4′-(chloro(phenyl)methylene)bis(methoxybenzene; DMTrt-Cl) (1.963 g, 5.8 mmol). After the addition of 4-(dimethylamino)-pyridine (0.046 g, 0.377 mmol), the reaction mixture was stirred at room temperature for 3 days. The reaction mixture was diluted with dichloromethane (100 mL) and water (40 mL). The organic phase was separated, dried over Na₂SO₄, filtered and the solvents were removed in vacuo. The residue was purified on HP-Sil SNAP cartridges (50 g) using a Biotage Isolera One purification system employing an ethyl acetate/n-heptane gradient (5/95->100/0->100/0) to afford the title compound C as a pale yellow solid (0.825 g, 72%). Unreacted starting material was recovered by flushing the cartridge with ethyl acetate/methanol (90/10) to afford the starting material as an off-white solid (0.042 g, 8.8%).

¹H-NMR (400 MHz, CDCl₃) δ=9.23 (s, 1H), 8.23 (d, 1H), 8.13 (d, 1H), 7.39-7.31 (m, 6H), 7.29-7.25 (4H), 6.80 (d, 4H), 6.41 (dd, 1H), 3.81 (s, 6H)

### Example 1

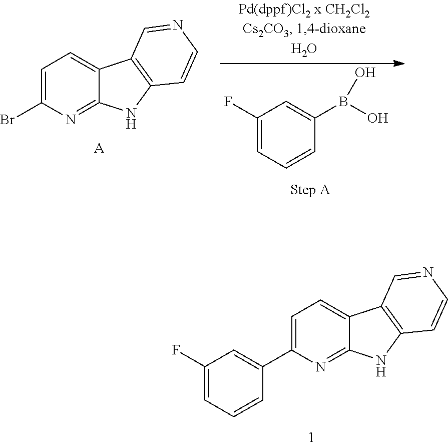

### Step A

To a mixture of degassed 1,4-dioxane (2.7 mL) and water (0.7 mL) in a microwave vial was added [1,1′-bis(diphenylphosphino)ferrocene]dichloropalladium(II) complex with dichloromethane (0.005 g, 0.006 mmol), followed by the title compound from Preparative Example A (0.03 g, 0.12 mmol), (3-fluorophenyl)boronic acid (0.021 g, 0.15 mmol) and cesium carbonate (0.08 g, 0.246 mmol). The reaction mixture was then heated at 115° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (12 g, puriFlash, Interchim) using a Biotage Isolera system employing a dichloromethane/methanol gradient (100/0->95/5->93/7->93/7) to afford the title compound 1 as an off-white solid (0.0091 g, 29%).

¹H-NMR (400 MHz, DMSO-d₆) 5=12.36 (br-s, 1H), 9.40 (s, 1H), 8.73 (d, 1H), 8.50 (d, 1H), 8.07 (d, 1H), 8.00 (d, 2H), 7.61-7.49 (m, 2H), 7.30 (dt, 1H)

MS (ESI): m/z=264.15 [M+H]⁺

### Examples 2 to 3a

Following the coupling procedure as described in Example 1, except using the boronic acid or ester derivatives indicated in the table below, the following compounds were prepared.

TABLE 1

| | Boronic | | 1. Yield |
|---|---|---|---|
| | acid/ester | Product | 2. ¹H-NMR |
| Bromo derivative | derivative | Example | 3. MH⁺ (ESI) |
| 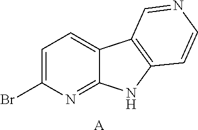 | 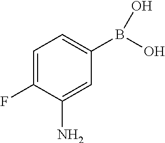 [SMILES: Nc1cc(B(O)O)ccc1F] | 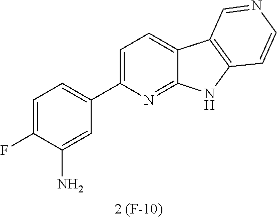 [SMILES: Nc1cc(-c2ccc3c(n2)[nH]c2ccncc23)ccc1F ; "2 (F-10)"] | 1. 37% 2. ¹H NMR (400 MHz, DMSO-d₆) δ = 12.25 (s, 1H), 9.36 (s, 1H), 8.65 (d, 1H), 8.48 (d, 1H), 7.77 (d, 1H), 7.64 (dd, 1H), 7.48 (d, 1H), 7.32 (m, 1H), 7.12 (dd, 1H), 5.32 (s, 2H) 3. 279.25 |
| 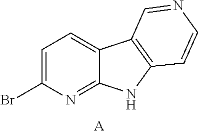 | 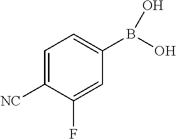 [SMILES: N#Cc1ccc(B(O)O)cc1F] | 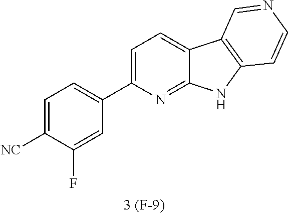 [SMILES: N#Cc1ccc(-c2ccc3c(n2)[nH]c2ccncc23)cc1F ; "3 (F-9)"] | 1. 35% 2. ¹H NMR (400 MHz, DMSO-d₆) δ = 12.38 (s, 1H), 9.41 (s, 1H), 8.76 (d, 1H), 8.70 (m, 1H), 8.61 (m, 1H), 8.51 (d, 1H), 8.05 (d, 1H), 7.71 (t, 1H), 7.51 (d, 1H) 3. 289.22 |
| 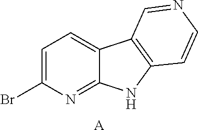 | 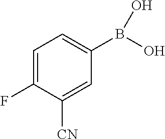 [SMILES: N#Cc1cc(B(O)O)ccc1F] | 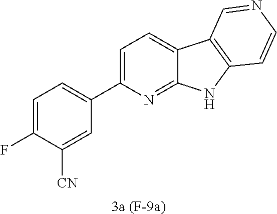 [SMILES: N#Cc1cc(-c2ccc3c(n2)[nH]c2ccncc23)ccc1F ; "3a (F-9a)"] | 1. 47% 2. ¹H NMR (400 MHz, DMSO-d₆) δ = 12.36 (s, 1H), 9.39 (s, 1H), 8.74 (d, 1H), 8.68 (d, 1H), 8.64-8.55 (m, 1H), 8.50 (d, 1H), 8.03 (d, 1H), 7.69 (t, 1H), 7.50 (d, 1H). 3. 288.93 |

### Example 4 (F-4)

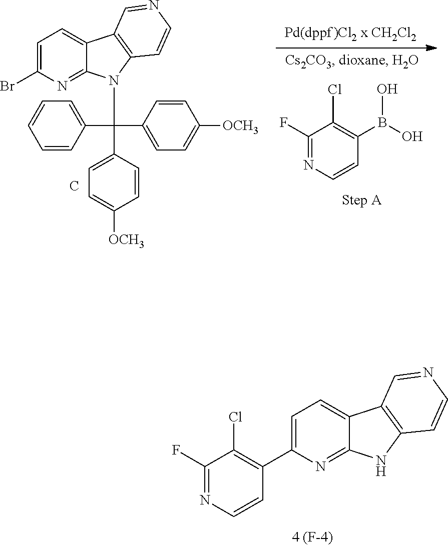

### Step A

To a mixture of degassed 1,4-dioxane (2.8 mL) and water (0.64 mL) in a microwave vial was added [1,1′-bis(diphenylphosphino)ferrocene]dichloropalladium(II) complex with dichloromethane (0.0053 g, 0.0064 mmol), followed by the title compound from Preparative Example C (0.07 g, 0.127 mmol), (2-fluoro-3-chloropyridin-4-yl)boronic acid (0.027 g, 0.156 mmol) and cesium carbonate (0.085 g, 0.26 mmol). The reaction mixture was then heated at ˜120° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (12 g, puriFlash, Interchim) using a Biotage Isolera system employing a dichloromethane/methanol gradient (100/0->95/5->90/10->80/20) to afford the title compound 4 as an off-white solid (0.0189 g, 50%).

¹H-NMR (400 MHz, DMSO-d₆) δ=12.52 (br-s, 1H), 9.47 (s, 1H), 8.83 (d, 1H), 8.55 (d, 1H), 8.34 (d, 1H), 7.75 (d, 1H), 7.72 (d, 1H), 7.53 (d, 1H)

MS (ESI): m/z=298.58 [M+H]⁺

### Example 5 (F-5)

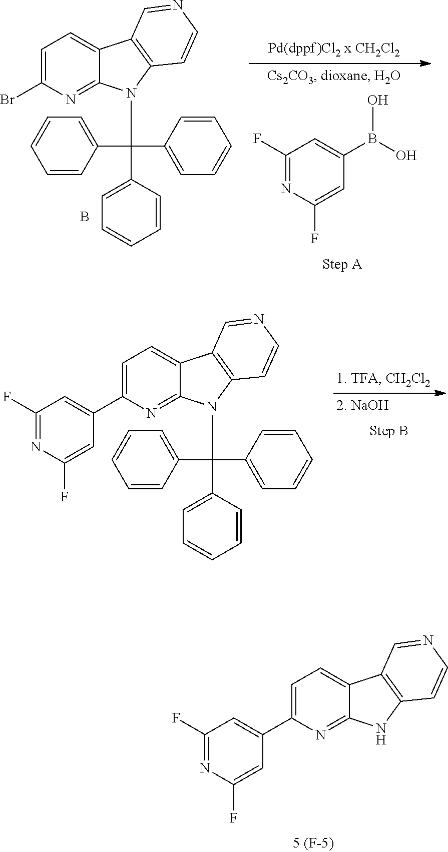

### Step A

To a mixture of degassed 1,4-dioxane (3.1 mL) and water (0.72 mL) in a microwave vial was added [1,1′-bis(diphenylphosphino)ferrocene]dichloropalladium(II) complex with dichloromethane (0.006 g, 0.0072 mmol), followed by the title compound from Preparative Example B (0.07 g, 0.148 mmol), (2,6-difluoropyridin-4-yl)boronic acid (0.028 g, 0.176 mmol) and cesium carbonate (0.096 g, 0.29 mmol). The reaction mixture was then heated at ˜120° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (12 g, puriFlash, Interchim) using a Biotage Isolera system employing an ethyl acetate/n-heptane gradient (5/95->100/0->100/0) to afford the title compound as a colorless glass (0.0687 g, 92%).

¹H-NMR (400 MHz, CDCl₃⁾ δ=9.30 (s, 1H), 8.46 (d, 1H), 8.29 (d, 1H), 7.74 (d, 1H), 7.58-7.54 (m, 5H); 7.32-7.27 (m, 10H), 6.86 (s, 2H), 6.62 (d, 1H)

### Step B

The title compound from Step A above (0.0687 g, 0.13 mmol) was dissolved in dichloromethane (5 mL). Trifluoroacetic acid (5 mL) was carefully added and the reaction mixture was stirred for 16 hours at room temperature. The solvents were evaporated under reduced pressure and the residue was dissolved in dichloromethane (50 mL) and water (20 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure. The residue was purified by chromatography on silica (12 g, puriFlash, Interchim) using a Biotage Isolera system employing a dichloromethane/methanol gradient (100/0->95/5->90/10->80/20) to afford the title compound 5 as a white solid (0.009 g, 25%).

¹H-NMR (400 MHz, DMSO-d₆) δ=12.51 (br-s, 1H), 9.45 (s, 1H), 8.85 (d, 1H), 8.54 (d, 1H), 8.22 (d, 1H), 7.93 (s, 2H), 7.52 (d, 1H)

MS [M+H]⁺=283.17

### Example 6 (F-7)

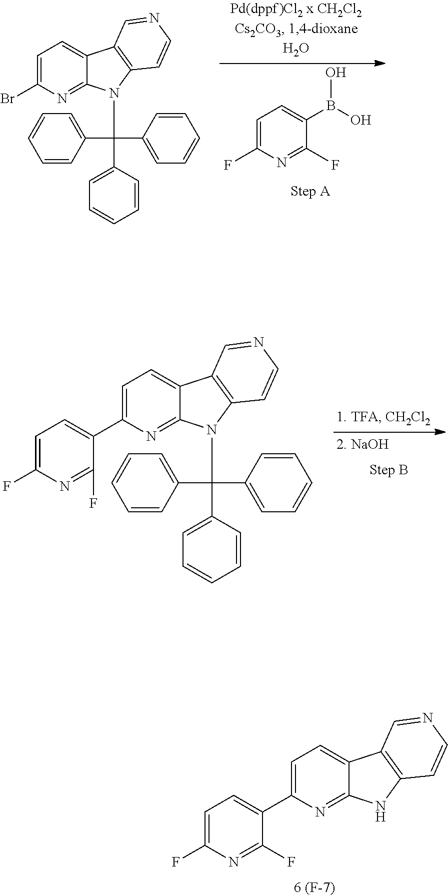

### Step A

To a mixture of degassed 1,4-dioxane (3.1 mL) and water (0.72 mL) in a microwave vial was added [1,1′-bis(diphenylphosphino)ferrocene]dichloropalladium(II) complex with dichloromethane (0.006 g, 0.0072 mmol), followed by the title compound from Preparative Example B (0.07 g, 0.148 mmol), (2,6-difluoropyridin-3-yl)boronic acid (0.028 g, 0.176 mmol) and cesium carbonate (0.096 g, 0.29 mmol). The reaction mixture was then heated at ˜120° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (25 g, puriFlash, Interchim) using a Biotage Isolera system employing an ethyl acetate/n-heptane gradient (5/95->100/0->100/0) to afford the title compound as an off-white solid (0.0606 g, 81%).

¹H-NMR (400 MHz, CDCl₃) δ=9.29 (s, 1H), 8.43 (d, 1H), 8.27 (d, 1H), 7.93 (d, 1H), 7.60-7.53 (m, 6H), 7.31-7.24 (m, 10H), 6.69 (dd, 1H), 6.59 (d, 1H)

MS [M+H]⁺=525.26

### Step B

The title compound from Step A above (0.0606 g, 0.114 mmol) was dissolved in dichloromethane (5 mL). Trifluoroacetic acid (2.5 mL) was carefully added and the reaction mixture was stirred for 16 hours at room temperature. The solvents were evaporated under reduced pressure and the residue was dissolved in dichloromethane (50 mL) and water (20 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure. The residue was purified by chromatography on silica (25 g, HP-SIL) using a Biotage Isolera system employing a dichloromethane/methanol gradient (100/0->95/5->90/10->80/20) to afford the title compound 6 as a white solid (0.0241 g, 75%).

¹H-NMR (400 MHz, DMSO-d₆) δ=12.46 (br-s, 1H), 9.43 (s, 1H), 8.79-8.73 (m, 2H), 8.54 (d, 1H), 7.82 (d, 1H), 7.53 (d, 1H), 7.40 (dd, 1H)

MS [M+H]⁺=283.15

### Example 7 (F-8)

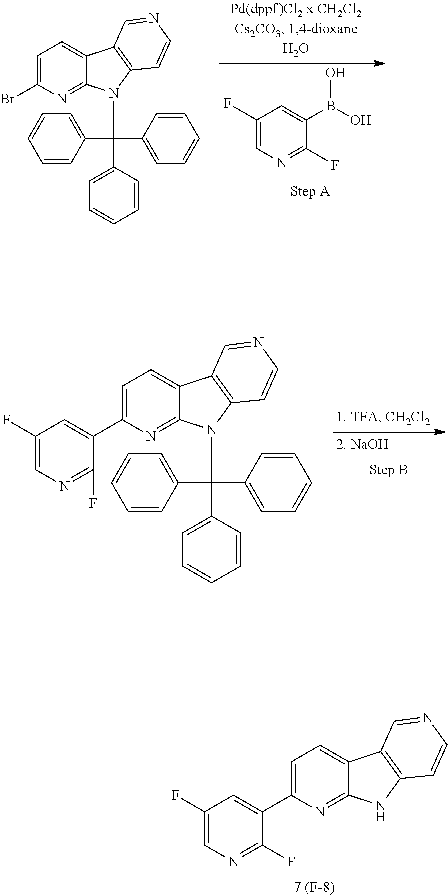

### Step A

To a mixture of degassed 1,4-dioxane (3.1 mL) and water (0.72 mL) in a microwave vial was added [1,1′-bis(diphenylphosphino)ferrocene]dichloropalladium(II) complex with dichloromethane (0.006 g, 0.0072 mmol), followed by the title compound from Preparative Example B (0.07 g, 0.148 mmol), (2,5-difluoropyridin-3-yl)boronic acid (0.028 g, 0.176 mmol) and cesium carbonate (0.096 g, 0.29 mmol). The reaction mixture was then heated at ˜120° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (25 g, puriFlash, Interchim) using a Biotage Isolera system employing an ethyl acetate/n-heptane gradient (5/95->100/0->100/0) to afford the title compound as a white solid (0.0554 g, 74%).

¹H-NMR (400 MHz, CDCl₃) δ=9.30 (s, 1H), 8.45 (d, 1H), 8.28 (d, 1H), 8.01 (d, 1H), 7.97 (t, 1H), 7.57-7.54 (m. 5H), 7.31-7.27 (m, 10H), 7.20 (td, 1H), 6.61 (d, 1H)

MS [M+H]⁺=525.24

### Step B

The title compound from Step A above (0.055 g, 0.105 mmol) was dissolved in dichloromethane (5 mL). Trifluoroacetic acid (2.5 mL) was carefully added and the reaction mixture was stirred for 16 hours at room temperature. The solvents were evaporated under reduced pressure and the residue was dissolved in dichloromethane (50 mL) and water (20 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure. The residue was purified by chromatography on silica (25 g, HP-SIL) using a Biotage Isolera system employing a dichloromethane/methanol gradient (100/0->95/5->90/10->80/20) to afford the title compound 7 as a white solid (0.0204 g, 71%).

¹H-NMR (400 MHz, DMSO-d₆) δ=12.48 (br-s, 1H), 9.44 (br-s, 1H), 8.80 (d, 1H), 8.55 (br-s, 1H), 8.48 (td, 1H), 8.38-8.36 (m, 1H), 7.88 (dd, 1H), 7.54 (d, 1H)

MS [M+H]⁺=283.21

### Example 8 (F-3)

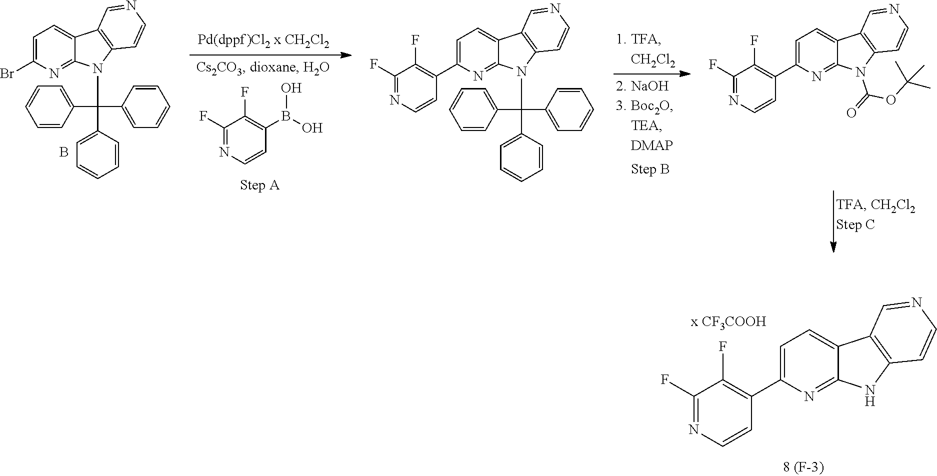

### Step A

To a mixture of degassed 1,4-dioxane (3.1 mL) and water (0.72 mL) in a microwave vial was added [1,1′-bis(diphenylphosphino)ferrocene]dichloropalladium(II) complex with dichloromethane (0.006 g, 0.0072 mmol), followed by the title compound from Preparative Example B (0.07 g, 0.148 mmol), (2,3-difluoropyridin-4-yl)boronic acid (0.028 g, 0.176 mmol) and cesium carbonate (0.096 g, 0.29 mmol). The reaction mixture was then heated at ˜120° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (12 g, puriFlash, Interchim) using a Biotage Isolera system employing an ethyl acetate/n-heptane gradient (5/95->100/0->100/0) to afford the title compound together with ˜10% of Preparative Example B as a colorless glass (0.0223 g, 29.7%).

Title compound: ¹H-NMR (400 MHz, CDCl₃) δ=9.31 (s, 1H), 8.46 (d, 1H), 8.29 (d, 1H), 7.99 (dd, 1H), 7.73 (dd, 1H), 7.57-7.53 (m, 5H), 7.32-7.24 (m, 10H), 6.85 (t, 1H), 6.56 (dd, 1H)

### Step B

The title compound from Step A above (0.0223 g, 0.0426 mmol) was dissolved in dichloromethane (2 mL) and trifluoroacetic acid (2 mL) was added. The reaction mixture was stirred at room temperature for 16 hours and then methanol was added (10 mL). The solvents were evaporated in vacuo and the residue was suspended in methanol (10 mL).

The solvents were again evaporated in vacuo and the residue was suspended in dichloromethane (3 mL). After the addition of triethylamine (1 mL, 7.7 mmol), di-tert-butyl dicarbonate (0.1 g, 0.43 mmol), and 4-(dimethylamino)-pyridine (0.0018 g, 0.014 mmol), the reaction mixture was stirred at room temperature for 18 hours. The reaction mixture was diluted with ethyl acetate (50 mL) and water (20 mL). The organic phase was separated, dried over Na₂SO₄, filtered and the solvents were removed in vacuo. The residue was purified on silica (25 g puriFlash, Interchim) using a Biotage Isolera One purification system employing an ethyl acetate/n-heptane gradient (5/95->100/0->100/0) to afford the title compound as an off-white solid (0.0062 g, 38%).

¹H NMR (400 MHz, CDCl₃) δ=9.39 (s, 1H), 8.77 (d, 1H), 8.55 (d, 1H), 8.37 (d, 1H), 8.17-8.11 (m, 3H), 1.85 (s, 9H)

MS (ESI): m/z=383.07 [MH]⁺

### Step C

Title compound from Step B above (0.0062 g, 0.016 mmol) was dissolved in dichloromethane (1 mL). Trifluoroacetic acid (1 mL) was carefully added and the reaction mixture was stirred for 16 hours at room temperature. The solvents were evaporated under reduced pressure and the residue was dissolved in methanol (5 mL). The solvents were evaporated in vacuo and the residue was dissolved in methanol (5 mL). The solvents were evaporated in vacuo to afford the TFA-salt of compound 8 as an off-white solid (0.0062 g, 97%).

¹H-NMR (400 MHz, DMSO-d₆) δ=13.88 (br-s, 1H), 9.89 (s, 1H), 9.07 (d, 1H), 8.80 (d, 1H), 8.25 (dd, 1H), 8.14 (dd, 1H), 8.06 (d, 1H), 8.00 (t, 1H)

MS [M+H]⁺=283.20

### Example 9 (F-6)

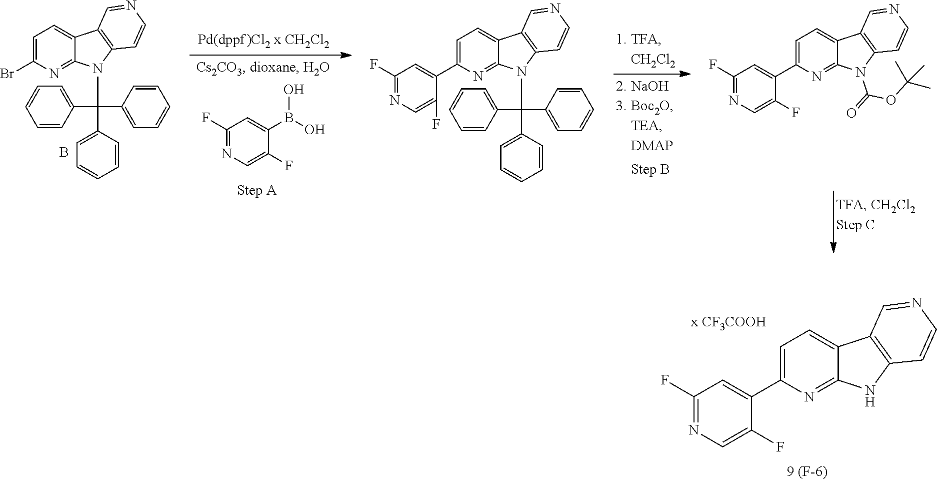

### Step A

To a mixture of degassed 1,4-dioxane (3.1 mL) and water (0.72 mL) in a microwave vial was added [1,1′-bis(diphenylphosphino)ferrocene]dichloropalladium(II) complex with dichloromethane (0.006 g, 0.0072 mmol), followed by the title compound from Preparative Example B (0.07 g, 0.148 mmol), (2,5-difluoropyridin-4-yl)boronic acid (0.028 g, 0.176 mmol) and cesium carbonate (0.096 g, 0.29 mmol). The reaction mixture was then heated at ˜120° C. in a sand-bath for 6 hours. The reaction mixture was diluted with ethyl acetate (60 mL) and water (20 mL), the organic phase was separated, dried over Na₂SO₄, filtered and the solvents were evaporated in vacuo. The dark residue was purified by chromatography on silica (12 g, puriFlash, Interchim) using a Biotage Isolera system employing an ethyl acetate/n-heptane gradient (5/95->100/0->100/0) to afford an inseparable mixture of the title compound and Preparative Example B (0.0578 g, 77%).

### Step B

The mixture from Step A above (0.0578 g, 0.11 mmol) was dissolved in dichloromethane (4 mL) and trifluoroacetic acid (4 mL) was added. The reaction mixture was stirred at room temperature for 16 hours and then methanol was added (10 mL). The solvents were evaporated in vacuo and the residue was suspended in methanol (10 mL). The solvents were again evaporated in vacuo and the residue was suspended in dichloromethane (6 mL). After the addition of triethylamine (2 mL, 14.4 mmol), di-tert-butyl dicarbonate (0.2 g, 0.86 mmol), and 4-(dimethylamino)-pyridine (0.0036 g, 0.028 mmol), the reaction mixture was stirred at room temperature for 18 hours. The reaction mixture was diluted with ethyl acetate (80 mL) and water (30 mL). The organic phase was separated, dried over Na₂SO₄, filtered and the solvents were removed in vacuo. The residue was purified on silica (25 g puriFlash, Interchim) using a Biotage Isolera One purification system employing an ethyl acetate/n-heptane gradient (5/95->100/0->100/0) to afford the title compound as an off-white solid (0.0141 g. 33%).

¹H NMR (400 MHz, CDCl₃) δ=9.39 (s, 1H), 8.77 (d, 1H), 8.54 (d, 1H), 8.39 (dd, 1H), 8.23 (dd, 1H), 8.15 (dd, 1H), 8.00 (dd, 1H), 1.84 (s, 3H)

MS (ESI): m/z=383.06 [MH]⁺

### Step C

Title compound from Step B above (0.0141 g, 0.037 mmol) was dissolved in dichloromethane (2 mL). Trifluoroacetic acid (2 mL) was carefully added and the reaction mixture was stirred for 16 hours at room temperature. The solvents were evaporated under reduced pressure and the residue was dissolved in methanol (5 mL). The solvents were evaporated in vacuo and the residue was dissolved in methanol (5 mL). The solvents were evaporated in vacuo to afford the TFA-salt of compound 9 as an off-white solid (0.0142 g, 97%).

¹H-NMR (400 MHz, DMSO-d₆) δ=13.84 (br-s, 1H), 9.88 (s, 1H), 9.06 (d, 1H), 8.80 (d, 1H), 8.52-8.51, (m, 1H), 8.13 (d, 1H), 8.05 (d, 1H), 7.82-7.80 (m, 1H)

MS [M+H]⁺=283.18

### Example 11 (F-11)

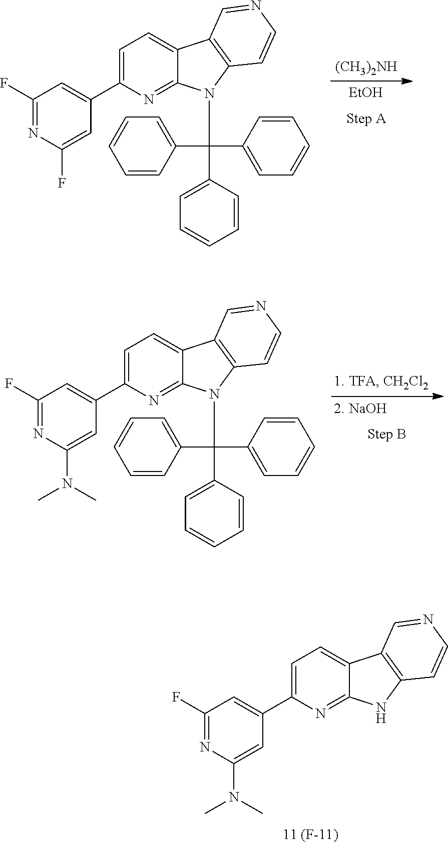

### Step A

The title compound from Example 5 Step A (0.138 g, 0.263 mmol) was dissolved/suspended in ethanol (1 mL). Then a 5.6 M solution of dimethylamine in ethanol (1.9 mL, 10.64 mmol) was added. The reaction mixture was then heated at 120° C. for 45 minutes using a Biotage Initiator microwave. The reaction mixture was diluted with ethyl acetate (80 mL) and water (20 mL). The organic phase was separated, dried over Na₂SO₄, filtered and the solvents were evaporated in vacuo. The dark residue was purified by chromatography on silica (25 g, puriFlash, Interchim) using a Biotage Isolera system employing an ethyl acetate/n-heptane gradient (5/95->100/0->100/0) to afford the title compound as a yellow solid (0.129 g, 89

¹H-NMR (400 MHz, CDCl₃) δ=9.26 (s, 1H), 8.37 (d, 1H), 8.25 (d, 1H), 7.67 (d, 1H), 7.59-7.54 (m, 6H), 7.29-7.21 (m, 9H), 6.58 (s, 1H), 6.31 (d, 1H), 6.26 (s, 1H), 3.02 (s, 6H)

MS (ESI): m/z=550.73 [M+H]⁺

### Step B

The title compound from Step A above (0.129 g, 0.235 mmol) was dissolved in dichloromethane (10.5 mL). Trifluoroacetic acid (5.25 mL) was carefully added and the reaction mixture was stirred for 18 hours at room temperature. The solvents were evaporated under reduced pressure and the residue was dissolved in dichloromethane (60 mL) and water (20 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure. The residue was purified by chromatography on silica (25 g, HP-Ultra) using a Biotage Isolera system employing a dichloromethane/methanol gradient (100/0->95/5->90/10->80/20) to afford the title compound 11 as an off-white solid (0.029 g, 40%).

¹H-NMR (400 MHz, DMSO-d₆) δ=12.46 (br-s, 1H), 9.42 (s, 1H), 8.76 (d, 1H), 8.52 (d, 1H), 8.07 (d, 1H), 7.49 (d, 1H), 7.25 (d, 1H), 6.96 (s, 1H), 3.13 (s, 6H)

MS (ESI): m/z=308.51 [M+H]⁺

### Example 12 (F-12)

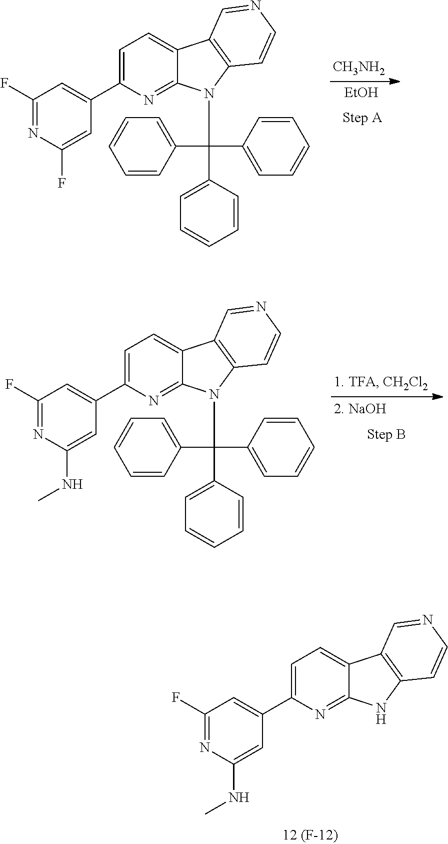 [SMILES: "EtOH" ; Fc1cc(-c2ccc3c4cnccc4n(C(c4ccccc4)(c4ccccc4)c4ccccc4)c3n2)cc(F)n1 ; "CH3NH2" ; CNc1cc(-c2ccc3c4cnccc4n(C(c4ccccc4)(c4ccccc4)c4ccccc4)c3n2)cc(F)n1 ; CNc1cc(-c2ccc3c(n2)[nH]c2ccncc23)cc(F)n1]

### Step A

The title compound from Example 5 Step A (0.075 g, 0.143 mmol) was dissolved/suspended in ethanol (1 mL). Then a 8 M solution of methylamine in ethanol (1.9 mL, 15.2 mmol) was added. The reaction mixture was then heated at 120° C. for 45 minutes using a Biotage Initiator microwave. The reaction mixture was diluted with ethyl acetate (80 mL) and water (20 mL). The organic phase was separated, dried over Na₂SO₄, filtered and the solvents were evaporated in vacuo. The dark residue was purified by chromatography on silica (25 g, puriFlash, Interchim) using a Biotage Isolera system employing an ethyl acetate/n-heptane gradient (5/95->100/0->100/0) to afford the title compound as a pale yellow solid (0.064 g, 84%).

¹H-NMR (400 MHz, CDCl₃) δ=9.26 (s, 1H), 8.38 (d, 1H), 8.27 (d, 1H), 7.67 (d, 1H), 7.61-7.57 (m, 5H), 7.29-7.22 (m, 10H), 6.49 (d, 1H), 6.32 (s, 2H), 4.53-4.49 (m, 1H), 2.90 (d, 3H)

MS (ESI): m/z=536.67 [M+H]⁺

### Step B

The title compound from Step A above (0.064 g, 0.12 mmol) was dissolved in dichloromethane (5.5 mL). Trifluoroacetic acid (2.6 mL) was carefully added and the reaction mixture was stirred for 18 hours at room temperature. The solvents were evaporated under reduced pressure and the residue treated with methanol (3 mL). The solvents were evaporated under reduced pressure, the residue dissolved in methanol (10 mL), and then added to a separation funnel containing dichloromethane (100 mL) and water (30 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure to obtain a solid material. The aqueous phase was decanted from the solid material, the solid material treated with methanol (15 mL), and the solvents evaporated under reduced pressure to obtain another batch of solid material. The combined solid material was purified by chromatography on silica (25 g, HP-Ultra) using a Biotage Isolera system employing a dichloromethane/methanol gradient (100/0->95/5->90/10->80/20) to afford the title compound 12 as an off-white solid (0.0125 g, 35 ¹H-NMR (400 MHz, DMSO-de) 5=12.40 (br-s, 1H), 9.42 (s, 1H), 8.75 (d, 1H), 8.52 (d, 1H), 7.94 (d, 1H), 7.50 (d, 1H), 7.14 (s, 1H), 7.11-7.07 (m, 1H), 6.85 (s, 1H), 2.83 (d, 3H)

MS (ESI): m/z 294.49 [M+H]⁺

### Example 13 (F-13)

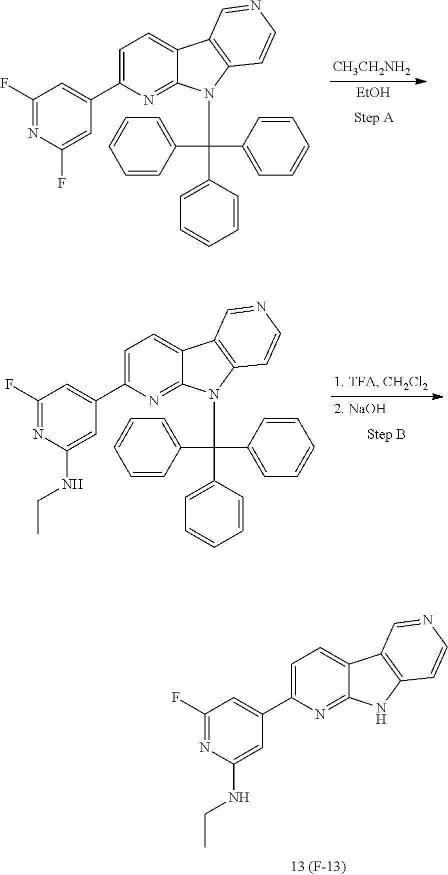

### Step A

The title compound from Example 5 Step A (0.1 g, 0.191 mmol) was dissolved/suspended in ethanol (1 mL). Then a 2 M solution of ethylamine in ethanol (2.5 mL, 5 mmol) was added. The reaction mixture was then heated at 120° C. for 45 minutes using a Biotage Initiator microwave. The reaction mixture was diluted with ethyl acetate (80 mL) and water (20 mL). The organic phase was separated, dried over Na₂SO₄, filtered and the solvents were evaporated in vacuo. The dark residue was purified by chromatography on silica (25 g, puriFlash, Interchim) using a Biotage Isolera system employing an ethyl acetate/n-heptane gradient (5/95->100/0->100/0) to afford the title compound (0.06 g) containing some starting material. The mixture was again purified by chromatography on silica (25 g, puriFlash, Interchim) using a Biotage Isolera system employing an ethyl acetate/n-heptane gradient (5/95->100/0->100/0) to afford the title compound as a pale yellow solid (0.041 g, 39%).

¹H-NMR (400 MHz, CDCl₃) δ=9.26 (s, 1H), 8.38 (d, 1H), 8.26 (d, 1H), 7.67 (d, 1H), 7.61-7.57 (m, 5H), 7.29-7.22 (m, 10H), 6.49 (d, 1H), 6.30 (s, 2H), 4.46-4.42 (m, 1H), 3.24 (q, 2H), 1.29 (td, 3H)

MS (ESI): m/z=550.69 [M+H]⁺

### Step B

The title compound from Step A above (0.041 g, 0.075 mmol) was dissolved in dichloromethane (3.6 mL). Trifluoroacetic acid (1.7 mL) was carefully added and the reaction mixture was stirred for 18 hours at room temperature. The solvents were evaporated under reduced pressure and the residue treated with methanol (3 mL). The solvents were evaporated under reduced pressure, the residue dissolved in methanol (10 mL), and then added to a separation funnel containing dichloromethane/methanol (9/1; 100 mL) and water (30 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure. The residue was purified by chromatography on silica (12 g, puriFlash, Interchim) using a Biotage Isolera system employing a dichloromethane/methanol gradient (100/0->95/5->90/10->80/20) to afford the title compound 13 as an off-white solid (0.0065 g, 28%).

¹H-NMR (400 MHz, DMSO-d₆) δ=12.39 (br-s, 1H), 9.41 (s, 1H), 8.75 (d, 1H), 8.52 (d, 1H), 7.93 (d, 1H), 7.51 (d, 1H), 7.16-7.12 (m, 2H), 6.83 (s, 1H), 3.33-3.32 (m, 2H), 1.18 (t, 3H)

MS (ESI): m/z=308.50 [M+H]⁺

### Example 14 (F-14)

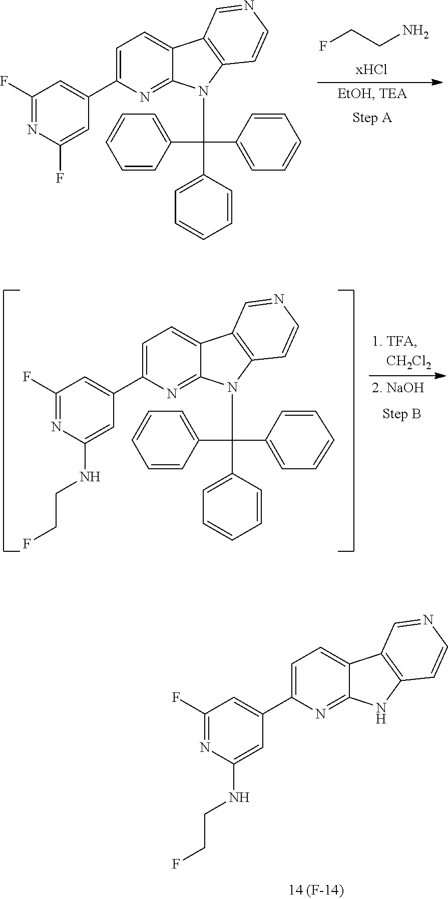

### Step A

The title compound from Example 5 Step A (0.1 g, 0.191 mmol) was dissolved/suspended in ethanol (3.5 mL). Then 2-fluoroethaneamine hydrochloric acid salt (0.378 g, 3.82 mmol) was added followed by trimethylamine (0.5 mL, 5 mmol). The reaction mixture was then heated at 120° C. for 45 minutes using a Biotage Initiator microwave. The reaction mixture was diluted with ethyl acetate (80 mL) and water (20 mL). The organic phase was separated, dried over Na₂SO₄, filtered and the solvents were evaporated in vacuo. The dark residue was purified by chromatography on silica (25 g, puriFlash, Interchim) using a Biotage Isolera system employing an ethyl acetate/n-heptane gradient (5/95->100/0->100/0) to afford a mixture of the title compound and starting material (0.02 g) which was directly used for the next step.

### Step B

The mixture from Step A above (0.02 g, 0.035 mmol) was dissolved in dichloromethane (1.5 mL). Trifluoroacetic acid (0.9 mL) was carefully added and the reaction mixture was stirred for 18 hours at room temperature. The solvents were evaporated under reduced pressure and the residue treated with methanol (3 mL). The solvents were evaporated under reduced pressure, the residue dissolved in methanol (10 mL), and then added to a separation funnel containing dichloromethane/methanol (9/1; 50 mL) and water (15 mL). The pH of the aqueous phase was adjusted to pH ˜12 by the addition of a 1 M aqueous sodium hydroxide solution. The organic phase was separated, dried over Na₂SO₄, filtered and the solvents were evaporated under reduced pressure. The residue was purified by chromatography on silica (12 g, puriFlash, Interchim) using a Biotage Isolera system employing a dichloromethane/methanol gradient (100/0->95/5->90/10->80/20) to afford the title compound 14 as an off-white solid (0.0021 g, 18%).

¹H-NMR (400 MHz, DMSO-de) 5=12.41 (br-s, 1H), 9.42 (s, 1H), 8.76 (d, 1H), 8.52 (d, 1H), 7.93 (d, 1H), 7.51 (dd, 1H), 7.44 (t, 1H), 7.25-7.24 (m, 1H), 6.89 (s, 1H), 4.64 (t, 1H), 4.52 (t, 1H), 3.66-3.62 (m, 1H), 3.59.3.55 (m, 1H)

MS (ESI): m/z=326.47 [M+H]⁺

### Example 15 (F-15)

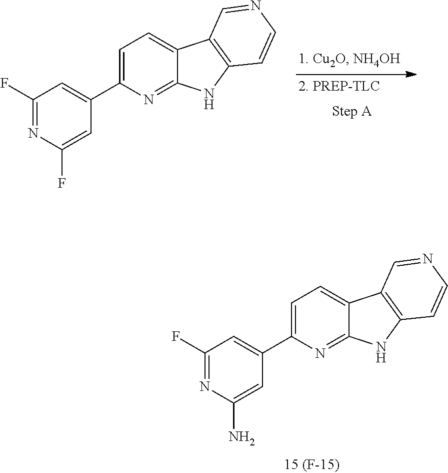 [SMILES: Fc1cc(-c2ccc3c(n2)[nH]c2ccncc23)cc(F)n1 ; Nc1cc(-c2ccc3c(n2)[nH]c2ccncc23)cc(F)n1]

### Step A

To the title compound from Example 5 (0.05 g, 0.18 mmol) was added a 32% aqueous ammonia solution (3.5 mL, 39.2 mmol) followed by copper(I)-oxide (0.004 g, 0.029 mmol). The reaction mixture was then heated at 145° C. for 1 hour using a Biotage Initiator microwave. The reaction mixture was diluted with water (10 mL), the precipitate collected by filtration, washed with water (2×5 mL) and dried under vacuum. The residue was purified by chromatography on silica (25 g, HP-Ultra) using a Biotage Isolera system employing a dichloromethane/methanol gradient (100/0->95/5->90/10->80/20->50/50->50/50). Fractions containing the title compound were collected, and the solvents evaporated under reduced pressure. The residue was purified by PREP-TLC (Analtech, 20×20 cm, 1000 μM) using dichloromethane/methanol (4/1) as mobile phase to afford the title compound 15 as off-white solid (0.0099 g, 20%).

¹H-NMR (400 MHz, DMSO-d₆) δ=12.46 (br-s, 1H), 9.42 (s, 1H), 8.76 (d, 1H), 8.53 (d, 1H), 7.91 (d, 1H), 7.53 (d, 1H), 7.16 (s, 1H), 6.86 (s, 1H), 6.54 (br-s, 2H)

MS (ESI): m/z=280.44 [M+H]⁺

### Determination of Tau Binding in AD Brain Homogenate

20 μg of human Alzheimer disease brain homogenate was incubated with a dilution series of each test compound (1000 to 0.06 nM) in the presence of 800 Bq of ¹⁸F-labeled Tau binder. The samples were shaken at 110 rpm for 45 min at 37° C. Samples were then filtered through GF/B 96 well filter plates and washed twice with 300 μL assay buffer (PBS containing 0.1% BSA and 2% DMSO). Thereafter, filter plates were sealed and a Fuji Film Imaging Plate (BAS-SR2025) was placed on top. The imaging plate was analyzed after overnight exposition using a Fuji Film BAS-5000. Non-specific signal was determined with samples containing ¹⁸F-labeled Tau-reference binder in the presence of assay buffer without brain substrate and competitor. Specific binding was calculated by subtracting the non-specific signal from the measured samples signal. The unblocked ¹⁸F-labeled Tau-binder signal was defined as total binding. IC₅₀ values were calculated by Prism V6 (GraphPad) setting total binding to 100%.

### Results:

High tau-affinity of compounds F-1, F-2, F-3, F-7, F-8, F9a and F-10 were found in a competition assay using human AD brain homogenate. IC₅₀ values for tau binding of 10 nM were measured.

### Determination of the Binding Affinity to Amyloid-Beta in AD Brain Homogenate

20 μg of human Alzheimer disease brain homogenate was incubated with a dilution series of each test compound (1000 to 0.06 nM) in the presence of 800 Bq of ¹⁸F-labeled beta-amyloid binder. The samples were shaken at 110 rpm for 45 min at 37° C. Samples were then filtered through GF/B 96-well filter plates and washed twice with 300 μL assay buffer (PBS containing 0.1% BSA and 2% DMSO). Thereafter, filter plates were sealed and a Fuji Film Imaging Plate (BAS-SR2025) was placed on top. The imaging plate was analyzed after overnight exposition using a Fuji Film BAS-5000. Non-specific signal was determined with samples containing ¹⁸F-labeled beta-amyloid binder in the presence of assay buffer without brain substrate and competitor. Specific binding was calculated by subtracting the non-specific signal from the measured samples signal. The unblocked ¹⁸F-labeled beta-amyloid binder signal was defined as total binding. IC₅₀ values were calculated by Prism V6 (GraphPad) setting total binding to 100%.

### Results:

Low affinity of the test compounds for beta-amyloid was found in a competition assay using human AD brain homogenate. IC₅₀ values for beta-amyloid binding of >500 nM were measured for all compounds.

### Determination of the Binding Affinity to MAO A in HC and Mouse Brain Homogenate

20 μg of brain homogenate (without AD pathology) was incubated with a dilution series of each test compound (1000 to 0.06 nM) in the presence of 800 Bq of 18F-labeled MAO-A binder ([¹⁸F]fluoroethyl harmine, FEH). The samples were shaken at 110 rpm for 45 min at 37° C. Samples were then filtered through GF/B 96-well filter plates and washed twice with 300 μL assay buffer (PBS containing 0.1% BSA and 2% DMSO). Thereafter, filter plates were sealed and a Fuji Film Imaging Plate (BAS-SR2025) was placed on top. The imaging plate was analyzed after overnight exposition using a Fuji Film BAS-5000. Non-specific signal was determined with samples containing ¹⁸F-labeled FEH in the presence of assay buffer without brain substrate and competitor. Specific binding was calculated by subtracting the non-specific signal from the measured samples signal. The unblocked ¹⁸F-labeled FEH signal was defined as total binding. IC₅₀ values were calculated by Prism V6 (GraphPad) setting total binding to 100%.

### Results:

In the mouse brain homogenate, compound F-1 showed a high off-target affinity towards MAO A of 22 nM in the ¹⁸F-FEH competition assay, and for compound F-2 of 475 nM, whereas off-target affinity to MAO A for e.g. compounds F-3, F-4, F-5, F-6 and F-8 was further reduced with IC₅₀ values of >1000 nM. Using human control brain homogenate (healthy control) compound F-1 showed a high off-target affinity towards MAO A of 5 nM in the FEH competition assay. The affinity of compound F-2 was reduced to 100 nM, whereas off-target affinity to MAO A for e.g. compounds F-3, F-4, F-5, F-6 and F-8 was further reduced with IC₅₀ values of >1000 nM each.

### Determination of the Binding Affinity to MAO B in HC Brain Homogenate

20 μg of human brain homogenate (without AD pathology) was incubated with a dilution series of each test compound (1000 to 0.06 nM) in the presence of 800 Bq of ¹⁸F-labeled MAO-B binder ([¹⁸F]fluoro deprenyl). The samples were shaken at 110 rpm for 45 min at 37° C. Samples were then filtered through GF/B 96-well filter plates and washed twice with 300 μL assay buffer (PBS containing 0.1% BSA and 2% DMSO). Thereafter, filter plates were sealed and a Fuji Film Imaging Plate (BAS-SR2025) was placed on top. The imaging plate was analyzed after overnight exposition using a Fuji Film BAS-5000. Non-specific signal was determined with samples containing ¹⁸F-labeled fluoro deprenyl in the presence of assay buffer without brain substrate and competitor. Specific binding was calculated by subtracting the non-specific signal from the measured samples signal. The unblocked ¹⁸F-labeled fluoro deprenyl signal was defined as total binding. IC₅₀ values were calculated by Prism V6 (GraphPad) setting total binding to 100%.

### Results:

In the human HC brain homogenate, compound F-1 showed a high off-target affinity towards MAO B of 170 nM in the ¹⁸F-labeled fluoro deprenyl competition assay. The affinity of e.g. compounds F-4, F-5, F-6, F-8, F-9 and F-10, was reduced to values >1000 nM, of compound F-3 to >600 nM.

As can be seen from Table 1, the prior art compounds F-1 and F-2 have limitations in respect to their affinity for MAO A and/or for MAO B, and thus low selectivity to Tau.

Due at least to its high affinity to Tau and/or lower binding affinity to other brain targets, compounds F-3 and F-8 have significantly better potential for determining and quantifying Tau deposits in the brain by positron emission tomography than the prior art compounds F-1 and F-2. In addition to the detection and quantification of Tau deposits in AD, compounds F-3 and F-8 can be useful for clinical evaluation of non-AD tauopathies.

TABLE 1
Summary of affinities determined as described in the experimental section above)

| | F-3 | F-4 | F-5 | F-6 | F-7 | F-8 | F-9 | F-9a | F-10 | F-1 | F-2 |
|---|---|---|---|---|---|---|---|---|---|---|---|
| High affinity to Tau | +++ | ++ | ++ | ++ | ++ | ++ | ++ | +++ | +++ | +++ | +++ |
| IC₅₀ in AD brain homogenate | 7 | 26 | 29 | 17 | 10 | 9 | 13 | 8.7 | 2 | 2 | 2 |
| | nM | nM | nM | nM | nM | nM | nM | nM | nM | nM | nM |
| Low affinity to MAO A | +++ | +++ | +++ | +++ | +++ | +++ | ++ | n.d. | + | − | + |
| IC₅₀ in HC homogenate | >1000 | >1000 | >1000 | >1000 | 739 | >1000 | 333 | | 24 | 5 | 100 |
| | nM | nM | nM | nM | nM | nM | nM | | nM | nM | nM |
| Low affinity to MAO A | +++ | +++ | +++ | +++ | +++ | +++ | ++ | ++ | + | + | ++ |
| IC₅₀ in mouse homogenate | 830 | >1000 | >1000 | >1000 | 701 | >1000 | 181 | 152 | 25 | 22 | 475 |
| | nM | nM | nM | nM | nM | nM | nM | nM | nM | nM | nM |
| Low affinity to MAO B | +++ | +++ | +++ | +++ | ++ | +++ | +++ | n.d. | +++ | + | +++ |
| IC₅₀ in HC brain homogenate | 645 | >1000 | >1000 | >1000 | 387 | >1000 | >1000 | | >1000 | 170 | >1000 |
| | nM | nM | nM | nM | nM | nM | nM | | nM | nM | nM |
| Low affinity to amyloid-beta | +++ | +++ | +++ | +++ | +++ | +++ | +++ | n.d. | +++ | +++ | +++ |
| IC₅₀ in AD brain homogenate | >1000 | >1000 | >1000 | >1000 | >1000 | >1000 | >1000 | | 680 | >1000 | >1000 |
| | nM | nM | nM | nM | nM | nM | nM | | nM | nM | nM |

− poor,
∘ moderate,
+ good,
++ very good,
+++ excellent,
n.d.: not determined
All data determined with the non-radioactive fluorine-19 derivatives

1. A compound of the formula (I) 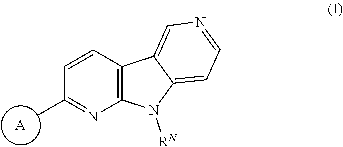
as well as pharmaceutically acceptable salts, hydrates, solvates, prodrugs and polymorphs thereof;
wherein 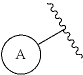
is selected from 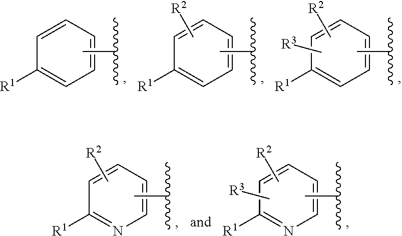
R¹ is ¹⁸F, ¹⁹F or LG,
R² and R³ are independently selected from the group consisting of halogen, alkyl, alkoxy, —NR⁷R⁸, —N(R⁷)alkyl, —N(alkyl)₂, and cyano, wherein the alkyl group(s) in alkyl, alkoxy, —N(R⁷)alkyl and —N(alkyl)₂ are independently optionally substituted with one or more halogen(s),
R⁷ and R⁸ are each independently selected from the group consisting of hydrogen and PG1,
R^{N} is hydrogen or PG2,
LG is a leaving group, and
PG1 and PG2 are independently selected from amine protecting groups.

2. The compound according to claim 1, wherein 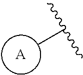
is selected from

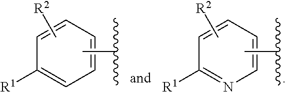

3. The compound according to claim 1, wherein
R² and R³ are independently selected from the group consisting of halogen, —NR⁷R⁸, —N(R⁷)alkyl, —N(alkyl)₂, and cyano, wherein the alkyl group(s) in —N(R⁷)alkyl and —N(alkyl)₂ are independently optionally substituted with one or more halogen(s).

4. The compound according to claim 1, wherein the compound has the formula (II) 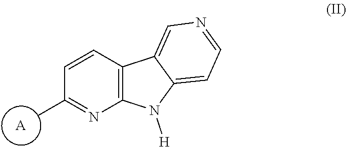
as well as pharmaceutically acceptable salts, hydrates, solvates, prodrugs and polymorphs thereof;
wherein 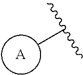
is selected from 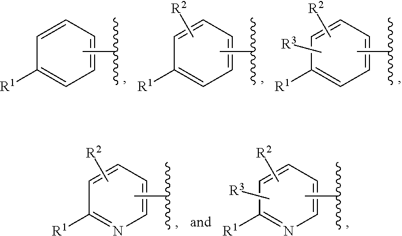
preferably 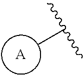
is selected from 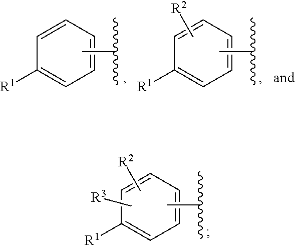
more preferably 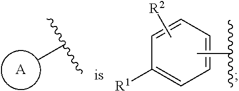
R¹ is ¹⁸F or ¹⁹F; preferably R¹ is ¹⁸F, and
R² and R³ are independently selected from the group consisting of halogen, alkyl, alkoxy, —NH₂, —N(H)alkyl, —N(alkyl)₂, and cyano, wherein the alkyl group(s) in alkyl, alkoxy, —N(H)alkyl and —N(alkyl)₂ are independently optionally substituted with one or more halogen(s).

5.-9. (canceled)

10. The compound according to claim 4, wherein the compound has the formula (IIa) 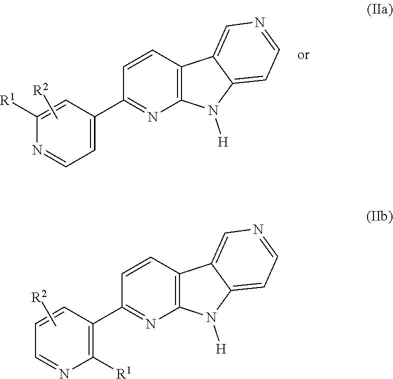
wherein R¹ and R² are as defined in claim 4.

11.-13. (canceled)

14. The compound according to claim 1, wherein the compound has the formula (III) 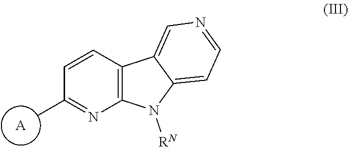
wherein 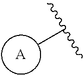
is selected from 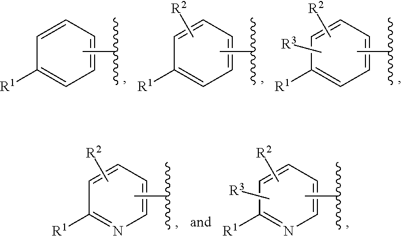
R¹ is LG,
R² and R³ are independently selected from the group consisting of halogen, alkyl, alkoxy, —NR⁷R⁸, —N(R⁷)alkyl, —N(alkyl)₂, and cyano, wherein the alkyl group(s) in alkyl, alkoxy, —N(R⁷)alkyl and —N(alkyl)₂ are independently optionally substituted with one or more halogen(s),
R⁷ and R⁸ are independently selected from the group consisting of hydrogen and PG1,
LG is a leaving group,
R^{N} is hydrogen or PG2, and
PG1 and PG2 are independently selected from amine protecting groups.

15. The compound according to claim 1, wherein the compound is detectably labeled, preferably wherein the detectable label is selected from 2H, 3H and 18F.

16.-17. (canceled)

18. A diagnostic composition comprising a compound as defined in claim 1 and optionally a pharmaceutically acceptable carrier, diluent, adjuvant or excipient.

19. (canceled)

20. A method of Tau aggregates, particularly a method of imaging Tau aggregates by positron emission tomography, wherein a diagnostically effective amount of a compound as defined in claim 1 is administered to a patient.

21. A method of diagnosing a disorder associated with Tau aggregates or a tauopathy, particularly wherein the diagnosis is conducted by positron emission tomography, wherein a diagnostically effective amount of a compound as defined in claim 1 is administered to a patient.

22.-23. (canceled)

24. A compound for use according to claim 21, wherein the disorder is selected from Alzheimer's disease (AD), familial AD, Creutzfeldt-Jacob disease, dementia pugilistica, Down's Syndrome, Gerstmann-Sträussler-Scheinker disease, inclusion-body myositis, prion protein cerebral amyloid angiopathy, traumatic brain injury (TBI), amyotrophic lateral sclerosis, Parkinsonism-dementia complex of Guam, non-Guamanian motor neuron disease with neurofibrillary tangles, argyrophilic grain disease, corticobasal degeneration (CBD), diffuse neurofibrillary tangles with calcification, frontotemporal dementia with Parkinsonism linked to chromosome 17, Hallervorden-Spatz disease, multiple system atrophy, Niemann-Pick disease type C, pallido-ponto-nigral degeneration, Pick's disease (PiD), progressive subcortical gliosis, progressive supranuclear palsy (PSP), subacute sclerosing panencephalitis, tangle only dementia, postencephalitic Parkinsonism, myotonic dystrophy, Tau panencephalopathy, AD-like with astrocytes, certain prion diseases (GSS with Tau), mutations in LRRK2, chronic traumatic encephalopathy, familial British dementia, familial Danish dementia, frontotemporal lobar degeneration, Guadeloupean Parkinsonism, neurodegeneration with brain iron accumulation, SLC9A6-related mental retardation, white matter tauopathy with globular glial inclusions, traumatic stress syndrome, epilepsy, Lewy body dementia (LBD), hereditary cerebral hemorrhage with amyloidosis (Dutch type), mild cognitive impairment (MCI), multiple sclerosis, Parkinson's disease, atypical parkinsonism, HIV-related dementia, adult onset diabetes, senile cardiac amyloidosis, endocrine tumors, glaucoma, ocular amyloidosis, primary retinal degeneration, macular degeneration (such as age-related macular degeneration (AMD)), optic nerve drusen, optic neuropathy, optic neuritis, lattice dystrophy, Huntington's disease, ischemic stroke and psychosis in AD preferably wherein the disorder is Alzheimer's disease (AD).

25.-26. (canceled)

27. An in vitro screening method, wherein a compound according to claim 1 is employed as an in vitro screening tool.

28. A method of preparing a compound as defined in claim 4 comprising reacting a compound as defined in claim 14 with a [¹⁸F]fluorinating agent, wherein the method further comprises cleaving of the protecting group PG1 and/or PG2, if present.

29. A kit for preparing a radiopharmaceutical preparation, said kit comprising a sealed vial containing a predetermined quantity of a compound as defined in claim 14.

30. A method of collecting data for the diagnosis of a disorder associated with tau aggregates in a sample or a patient comprising:
(a) bringing a sample or a specific body part or body area suspected to contain a tau aggregate into contact with a compound as defined in claim 1;
(b) allowing the compound to bind to the tau aggregate;
(c) detecting the compound bound to the tau aggregate; and
(d) optionally correlating the presence or absence of compound binding with the tau aggregate with the presence or absence of tau aggregate in the sample or specific body part or body area.

31. A method which comprises the steps of:
(a) bringing the sample suspected to contain the tau aggregate into contact with the compound as defined in claim 1, which compound specifically binds to the tau aggregate;
(b) allowing the compound to bind to the tau aggregate to form a compound/tau aggregate complex;
(c) detecting the formation of the compound/tau aggregate complex;
(d) optionally correlating the presence or absence of the compound/tau aggregate complex with the presence or absence of tau aggregate in the sample; and
(e) optionally comparing the amount of the compound/tau aggregate to a normal control value,
wherein the method is
a method of collecting data for determining a predisposition to a disorder associated with tau aggregates in a patient comprising detecting the specific binding of a compound as defined in claim 1 to a tau aggregate in a sample, or
a method of collecting data for predicting responsiveness of a patient suffering from a disorder associated with tau aggregates and being treated with a medicament.

32. (canceled)
